(19) United States
(12) Patent Application Publication
(10) Pub. No.: US 2005/0118639 A1
(43) Pub. Date: Jun. 2, 2005

(54) Method of determining ligand

(75) Inventors: Shuji Hinuma, Ibaraki (JP); Ryo Fujii, Ibaraki (JP); Kazuhiro Ogi, Ibaraki (JP); Hidetoshi Komatsu, Ibaraki (JP); Yuji Kawamata, Ibaraki (JP); Masaki Hosoya, Ibaraki (JP)

Correspondence Address: EDWARDS & ANGELL, LLP, P.O. BOX 55874, BOSTON, MA, 02205, US

(73) Assignee: Takeda Chemical Industries, Ltd, Osaka (JP)

(21) Appl. No.: 10/505,486

(22) PCT Filed: Feb. 21, 2003
(86) PCT No.: PCT/JP03/01901

(30) Foreign Application Priority Data
Feb. 22, 2002 (JP) 2002-045728
Jul. 23, 2002 (JP) 2002-213949
Oct. 11, 2002 (JP) 2002-298237

(51) Int. Cl. G01N 33/53; C07K 14/705

(52) U.S. Cl. 435/7.1; 530/350

(57) ABSTRACT

The present invention aims at providing a method of determining a ligand to an orphan receptor. Specifically, the present invention provides a method of determining a ligand to a receptor protein, to which no ligand has been determined, which comprises using a fusion protein of the receptor protein and a fluorescent protein.

### FIELD OF THE INVENTION

The present invention relates to a method of determining a ligand to a receptor protein present on cell membrane and in particular, relates to a method of determining a ligand to a so-called orphan receptor, of which the ligand is totally unknown.

### BACKGROUND ART

Physiologically active substances such as various hormones and neurotransmitters regulate the biological function through specific receptor proteins present on cell membranes. Many of these receptor proteins are coupled with guanine nucleotide-binding protein (hereinafter sometimes simply referred to as G protein) and mediate the intracellular signal transduction through activation of G protein. These receptor proteins have the common structure containing seven transmembrane domains and are thus collectively referred to as G protein-coupled receptor proteins or seven-transmembrane receptor proteins (7TMR).

G protein-coupled receptor proteins are present on the cell surface of each functional cell and organ in an organism, and play important physiological roles as the target of the molecules that regulate the functions of the cells and organs, e.g., hormones, neurotransmitters, physiologically active substances or the like. Receptors transmit signals to cells through binding with physiologically active substances, and the signals induce various reactions such as activation or suppression of the cells.

To clarify the relationship between substances that regulate the functions of cells and organs in various organisms, and their specific receptor proteins, in particular, G protein-coupled receptor proteins would elucidate the functions of cells and organs in various organisms to provide a very important means for development of pharmaceuticals closely associated with the functions.

Substances that inhibit binding of G protein-coupled receptor proteins to physiologically active substances (i.e., ligands) or substances that bind and induce signal transduction similar to that induced by physiologically active substances (i.e., ligands) have been hitherto used for pharmaceuticals, as antagonists and agonists specific to these receptors that regulate the biological functions.

However, even at the present moment, there are a large number of so-called orphan receptors in which the corresponding ligands are yet unidentified. Thus, it has been earnestly desired to identify the ligands and elucidate their functions.

G protein-coupled receptors are useful in searching for a novel physiological active substance (i.e., ligand) using the signal transduction activity as the index and in search for agonists and antagonists to the receptor. Ligands, agonists, antagonists, or the like to these receptors are expected to be used as prophylactic and/or therapeutic and diagnostic agents for diseases associated with dysfunction of the G protein-coupled receptors.

GFP (Green Fluorescent Protein) is one of fluorescent proteins derived from the jellyfish Aequorea Victoria of mesozoan animals (WO 96/23810, WO 96/27675, WO 97/26333, WO 97/28261, WO 97/42320).

A method of determining a ligand to Gi-, Gs- or Gq-coupled orphan receptor using multiple response elements (MRE) and a cAMP response element (CRE) in combination with a reporter activity is reported (Analytical Biochemistry, 275, 54-61, 1999).

A method of screening molecules which interact on Gs- or Gq-coupled orphan receptors using a cAMP response element (CRE) in combination with a reporter activity is reported (Analytical Biochemistry, 226, 349-354, 1995).

A method of characterization for Gi- or Gs-coupled receptors with known ligands using a cAMP response element (CRE) in combination with a reporter activity is reported (Current Opinion in Biotechnology, 1995, 6: 574-581).

A method of detecting the response of a G protein-coupled receptor using a cell in which chimera G protein a subunits with increased promiscuity in coupling specificity for the G protein-coupled receptor, chimera G protein has been expressed is reported (WO 01/36481).

A method of identifying a ligand for an orphan G protein-coupled receptor using a recombinant yeast expression library is reported (U.S. Pat. No. 6,255,059).

A method of identifying a receptor activity modifying substance composed of a cell containing both a receptor and a test peptide, which cell is useful for identifying or screening an orphan receptor is reported (US 2001/0026926).

An expression system for identifying a ligand to an orphan receptor utilizing the gamogenesis system of Saccharomyces is reported (WO 00/031261).

In conventional methods for searching/identifying a ligand, for example, when a ligand is screened using an eukaryotic cell, a so-called stable cell line capable of stably expressing its receptor should be established, and a special cell was required for establishing the cell line. Moreover, it was necessary for the screening to use a plurality of assays in combination. Thus, when a plurality of test compounds existed, it took a long time, which made it difficult to assay them. That is, conventional methods for searching/identifying ligands, etc. encounter problems that (1) a usable cell line is restricted, (2) it takes time to establish the cell line, (3) because of using a plurality of assays in combination, the number of specimens increases so that it becomes difficult to implement the methods, and so on. A method of determining a ligand that solves these problems and can use various cell lines and implement in a short period of time is desired.

### DISCLOSURE OF THE INVENTION

The present inventors made extensive investigations. As a result, by preparing a cell wherein a fusion protein of a receptor protein, to which no ligand has been determined, and a fluorescent protein such as GFP was stably or transiently expressed, the inventors have found (1) to (5) below, using immunostaining assay, western blotting assay, etc. utilizing fluorescence from a fluorescent protein such as GFP or a fluorescent protein antibody such as a GFP antibody that:
(1) expression of the receptor protein could be confirmed on a protein level;
(2) expression of the receptor protein on a cell membrane could be confirmed;
(3) an expression level of the receptor protein could be approximated;
(4) a cell with the receptor protein being highly expressed could be screened; and,
(5) a specific reaction of the receptor with the ligand could be detected as intracellular internalization of the fusion protein of the receptor and the fluorescent protein; etc. By utilizing these characteristics, the inventors found that a ligand to the receptor protein for which a ligand has not been identified (hereinafter sometimes simply referred to as an orphan receptor) could efficiently be determined. Based on these findings, the present inventors have made further investigations and come to accomplish the present invention.

That is, the present invention relates to the following features:
[1] A method of determining a ligand to a receptor protein for which a ligand has not been identified, which comprises using a fusion protein of the receptor protein with a fluorescent protein;
[2] The ligand determination method according to [1], wherein the fusion protein of a receptor protein for which a ligand has not been identified and GFP is used;
[3] The ligand determination method according to [1], wherein a cell expressed with the fusion protein of a receptor protein for which a ligand has not been identified and GFP or a membrane fraction of the cell;
[4] The ligand determination method according to [1], which comprises assaying (1) an activity that accelerates or suppresses arachidonic acid release, acetylcholine release, intracellular Ca²⁺ release, intracellular cAMP production, intracellular cGMP production, inositol phosphate production, changes in cell membrane potential, phosphorylation of intracellular proteins, activation of c-fos or pH reduction, (2) MAP kinase activation, (3) transcription factor activation, (4) diacylglycerol production, (5) opening or closing of ion channels on a cell membrane, (6) apoptosis induction, (7) morphological changes in cells, (8) transport of the fusion protein from cell membrane to cytoplasm, (9) low molecular weight G protein activation, (10) cell divisioh-promoting activity or (11) DNA synthesis-promoting activity;
[5] The ligand determination method according to [1], wherein transport of the fusion protein from cell membrane to cytoplasm is assayed;
[6] The ligand determination method according to [5], wherein transport of the fusion protein from cell membrane to cytoplasm is assayed by observing the fluorescence of GFP;
[7] The ligand determination method according to [1], which comprises bringing a cell capable of expressing the fusion protein of a receptor protein for which a ligand has not been identified the receptor protein and a fluorescent protein and containing a plasmid ligated with a DNA encoding a reporter protein at the downstream of cAMP response element/promoter in contact with a test compound and assaying the activity of the reporter protein;
[8] The method according to [7], which comprises culturing a cell containing (1) a plasmid containing a DNA encoding the fusion protein of a receptor protein for which a ligand has not been identified and a fluorescent protein and (2) a plasmid ligated with a DNA encoding a reporter protein at the downstream of cAMP response element/promoter, bringing the cell in contact with a test compound and assaying the activity of the reporter protein;
[9] The ligand determination method according to [2], wherein a cell capable of expressing the fusion protein of a receptor protein for which a ligand has not been identified and GFP and containing a plasmid ligated with a DNA encoding a reporter protein at the downstream of cAMP response element/promoter and assaying the activity of the reporter protein;
[10] The ligand determination method according to [9], which comprises culturing a cell containing (1) a plasmid containing a DNA encoding the fusion protein of a receptor protein for which a ligand has not been identified and GFP and (2) a plasmid ligated with a DNA encoding a reporter protein at the downstream of cAMP response element/promoter, bringing the cell in contact with a test compound and assaying the activity of the reporter protein;
[11] The method according to [1], wherein the receptor protein is a G protein-coupled receptor protein;
[12] The method according to [1], wherein GFP is a protein containing the same or substantially the same amino acid sequence as the amino acid sequence represented by SEQ ID NO: 1, SEQ ID NO: 3, SEQ ID NO: 5 or SEQ ID NO: 7;
[13] The method according to [7], wherein the promoter is a TATA-like sequence;
[14] The method according to [7], wherein the reporter protein is luciferase;
[15] The method according to [7], wherein the plasmid is a plasmid ligated with a TATA-like promoter and a gene encoding the reporter protein at the downstream of cAMP response element;
[16] The method according to [7], wherein the cell has expressed at least two receptor proteins for which ligand s have not been identified;
[17] The method according to [7], wherein the cell further contains a plasmid containing a gene encoding an inhibitory G protein α subunit Gi;
[18] The method according to [7], wherein forskolin is further added;
[19] The method according to [16], wherein the at least two receptor proteins have similar characteristics;
[20] The method according to [19], wherein the similar characteristics are the basic expression level of the reporter protein and/or the expression level of the reporter protein when forskolin is added;
[21] The method according to [16], which comprises measuring the basic expression level of the reporter protein when the at least two receptor proteins are previously expressed individually and/or the expression level of the reporter protein by the addition of forskolin and being expressed in combination the at least two receptor proteins having an equivalent expression level of the reporter protein;
[22] A fusion protein of a receptor protein for which a ligand has not been identified and a fluorescent protein, or a salt thereof;
[23] The fluorescent protein or a salt thereof according to [22], wherein the fluorescent protein is GFP;
[24] A DNA containing a DNA encoding the fusion protein according to [22];
[25] A recombinant vector containing the DNA according to [22];
[26] A transformant transformed with the recombinant vector according to [25];
[27] Use of a fluorescent protein to determine a ligand to a receptor protein for which a ligand has not been identified; and,
[28] Use of GFP to determine a ligand to a receptor protein for which a ligand has not been identified.

### BRIEF DESCRIPTION OF THE DRAWINGS

FIG. 1 shows the results (n=2) of detection of reporter activation by cholesterol metabolism-related substances in HEK293 cells transfected with TGR5 expression vector (A) and HEK293 cells transfected with intact vector alone (B).

FIG. 2 shows the results of induced expression of reporter genes by ligand stimulation in HEK293 cells.

FIG. 3 shows the results of TGR5 response to lithocholic acid in the co-presence of Gi.

FIG. 4 shows localization of the TGR5-GFP fusion protein expressed in CHO cells in the absence of ligand. In the figure, white line shows the length of 4 μm.

FIG. 5 shows localization of the fusion protein 30 minutes after the addition of TLCA to TGR5-GFP-expressed CHO cells. In the figure, white line shows the length of 4 μm.

### BEST MODE FOR CARRYING OUT THE INVENTION

The ligand determination method of the present invention is characterized by using (1) a fusion protein of a receptor protein for which a ligand has not been identified and a fluorescent protein, more specifically, is a method which comprises bringing a cell wherein the fusion protein of a receptor protein for which a ligand has not been identified and a fluorescent protein has been expressed or a membrane fraction of the cell in contact with a test compound (ligand determination method A).

Furthermore, the ligand determination method of the present invention includes (1) a method which involves bringing a cell capable of expressing a fusion protein of a receptor protein for which a ligand has not been identified and a fluorescent protein and containing a plasmid ligated with DNA encoding a reporter protein at the downstream of enhancer/promoter in contact with a test compound and assaying the activity of the expression-induced reporter protein, and (2) a method which involves culturing a cell containing (i) a plasmid containing DNA encoding a fusion protein of a receptor protein for which a ligand has not been identified and a fluorescent protein and (ii) a plasmid ligated with DNA encoding a reporter protein at the downstream of enhancer/promoter, and bringing the cell in contact with a test compound and assaying the activity of the expression-induced reporter protein (ligand determination method B).

As the orphan receptors used in the present invention, for example, G protein-coupled receptor proteins, etc. are employed. Specific examples include G protein-coupled receptor proteins containing the same or substantially the same amino acid sequence as the amino acid sequence represented by SEQ ID NO: 9, G protein-coupled receptor proteins described in WO 96/05302, EP-A-711831, EP-A-789076, EP-A-1103563, EP-A-1103562, Published Japanese Patent Application KOKAI Nos. 8-154682, 8-283295, 8-196278, 8-245697, 8-266280, 9-51795, 9-121865, 9-2388686 and 10-146192, and the like.

The fluorescent proteins are not particularly limited as far as it is visually recognizable, and include, e.g., GFP (Green Fluorescent Protein), Tag sequence, EGFP (enhanced green fluorescent protein), ECFP (enhanced cyan fluorescent protein), EYFP (enhanced yellow fluorescent protein), DsRED (Discosoma sp. red fluorescent protein), EBFP (enhanced blue fluorescent protein), etc.

GFP is one of fluorescent proteins derived from the jellyfish Aequorea Victoria of mesozoan animals, and includes proteins containing the same or substantially the same amino acid sequence as the amino acid sequence represented by SEQ ID NO: 1, SEQ ID NO: 3, SEQ ID NO: 5 or SEQ ID NO: 7, etc.

The amino acid sequence which is substantially the same amino acid sequence as the amino acid sequence represented by SEQ ID NO: 1, SEQ ID NO: 3, SEQ ID NO: 5 or SEQ ID NO: 7 includes an amino acid sequence having at least about 70% homology, preferably at least about 80% homology, more preferably at least about 90% homology and most preferably at least about 95% homology, to the amino acid sequence represented by SEQ ID NO: 1, SEQ ID NO: 3, SEQ ID NO: 5 or SEQ ID NO: 7; or the like.

Preferred examples of the protein of the present invention which contains substantially the same amino acid sequence as the amino acid sequence represented by SEQ ID NO: 1, SEQ ID NO: 3, SEQ ID NO: 5 or SEQ ID NO: 7 include a protein having substantially the same amino acid sequence as the amino acid sequence represented by SEQ ID NO: 1, SEQ ID NO: 3, SEQ ID NO: 5 or SEQ ID NO: 7 and having the activity substantially equivalent to proteins comprising the amino acid sequence represented by SEQ ID NO: 1, SEQ ID NO: 3, SEQ ID NO: 5 or SEQ ID NO: 7; and the like.

The activities of GFP include, e.g., fluorescence by excitation light irradiation, etc. The term substantially equivalent activities are used to mean that these activities are equivalent in nature. Thus, though it is preferred that these activities are equivalent (e.g., about 0.01- to about 100-fold, preferably about 0.5- to about 20-fold, more preferably about 0.5- to about 2-fold), quantitative factors such as an activity level, a molecular weight of the protein, etc. may be different.

The activities in GFP such as fluorescence by excitation light irradiation, etc. can be assayed by publicly known methods with modifications.

Proteins containing the following amino acid sequences are used as GFP: (1) the amino acid sequences wherein at least 1 or 2 amino acids (preferably approximately 1 to 30 amino acids, more preferably approximately 1 to 10 amino acids, most preferably several (1 to 5) amino acids) are deleted in the amino acid sequence represented by SEQ ID NO: 1, SEQ ID NO: 3, SEQ ID NO: 5 or SEQ ID NO: 7; (2) the amino acid sequences wherein at least 1 or 2 amino acids (preferably approximately 1 to 30 amino acids, more preferably approximately 1 to 10 amino acids, and most preferably several (1 to 5) amino acids) are added to or inserted into the amino acid sequence represented by SEQ ID NO: 1, SEQ ID NO: 3, SEQ ID NO: 5 or SEQ ID NO: 7; (3) the amino acid sequences wherein at least 1 or 2 amino acids (preferably approximately 1 to 30 amino acids, more preferably approximately 1 to 10 amino acids, and most preferably several (1 to 5) amino acids) are substituted by other amino acids in the amino acid sequence represented by SEQ ID NO: 1, SEQ ID NO: 3, SEQ ID NO: 5 or SEQ ID NO: 7; or (4) the amino acid sequence wherein these deletion, addition and substitution are combined; etc. Among others, preferably used are proteins containing (i) the amino acid sequence wherein the N-terminal methionine residue is deleted in the amino acid sequence represented by SEQ ID NO: 1, SEQ ID NO: 3, SEQ ID NO: 5 or SEQ ID NO: 7, (ii) the amino acid sequence wherein the N-terminal methionine residue is deleted and the following alanine residue is substituted with a threonine residue or serine residue in the amino acid sequence represented by SEQ ID NO: 1, SEQ ID NO: 3, SEQ ID NO: 5 or SEQ ID NO: 7; etc.

Specifically, there are used, for example, (i) GFP consisting of amino acid sequences represented by SEQ ID NO: 1, SEQ ID NO: 3, SEQ ID NO: 5 or SEQ ID NO: 7, (ii) GFP consisting of amino acid sequences represented by SEQ ID NO: 1, SEQ ID NO: 3, SEQ ID NO: 5 or SEQ ID NO: 7, wherein the N-terminal methionine residue is deleted, (iii) GFP consisting of amino acid sequences represented by SEQ ID NO: 1, wherein the N-terminal methionine residue is deleted and the following alanine residue is substituted with a threonine residue or serine residue; etc.

As the Tag sequence, for example, known sequences below are employed.

(SEQ ID NO: 17)

(1) His-tag (PCDNA3.1/His A)
His His His His His His

(SEQ ID NO: 18)

(2) V5-tag (PCDNA3.1/V5-His A)
Gly Lys Pro Ile Pro Asn Pro Leu Leu Gly Leu Asp
Ser Thr

(SEQ ID NO: 19)

(3) myc-tag (pCDNA3.1/myc-His A)
Glu Gln Lys Leu Ile Ser Glu Glu Asp Leu

(SEQ ID NO: 20)

(4) Xpress-tag (PCDNA3.1/His A)
Asp Leu Tyr Asp Asp Asp Asp Lys

(SEQ ID NO: 21)

(5) HA-tag (PCluzHA Expression vector)
Met Gly Ser Tyr Pro Tyr Asp Val Pro Asp Tyr Ala
Ser Leu Glu Phe

As EGFP, there is used a protein consisting of the amino acid sequence represented by SEQ ID NO: 7.

As ECFP, there is used a protein consisting of the amino acid sequence represented by SEQ ID NO: 22.

As EYFP, there is used a protein consisting of the amino acid sequence represented by SEQ ID NO: 24.

As DsRtD, there is used a protein consisting of the amino acid sequence represented by SEQ ID NO: 26.

As EBFP, there is used a protein consisting of the amino acid sequence represented by SEQ ID NO: 28.

These EGFP, ECFP, EYFP, DsRED and EBFP may be proteins having (1) the amino acid sequence wherein at least 1 or 2 amino acids (preferably approximately 1 to 30 amino acids, more preferably approximately 1 to 10 amino acids, most preferably several (1 to 5) amino acids) are deleted in the amino acid sequences described above; (2) the amino acid sequence wherein at least 1 or 2 amino acids (preferably approximately 1 to 30 amino acids, more preferably approximately 1 to 10 amino acids, and most preferably several (1 to 5) amino acids) are added to or inserted into the amino acid sequences described above; (3) the amino acid sequence wherein at least 1 or 2 amino acids (preferably approximately 1 to 30 amino acids, more preferably approximately 1 to 10 amino acids, and most preferably several (1 to 5) amino acids) are substituted by other amino acids in the amino acid sequences described above; or (4) the amino acid sequence wherein these deletion, addition and substitution are combined; etc., so long as they have the activities such as fluorescence by excitation light irradiation, etc.

Specifically, the DNA encoding GFP may be any DNA, so long as it is, for example, DNA containing the base sequence represented by SEQ ID NO: 2, SEQ ID NO: 4, SEQ ID NO: 6 or SEQ ID NO: 8, or DNA having a base sequence hybridizable to a complementary base sequence to the base sequence represented by SEQ ID NO: 2, SEQ ID NO: 4, SEQ ID NO: 6 or SEQ ID NO: 8 under high stringent conditions and encoding a protein which has the activities (e.g., fluorescence by excitement light irradiation, etc.) substantially equivalent to those of GFP consisting of the amino acid sequence represented by SEQ ID NO: 1, SEQ ID NO: 3, SEQ ID NO: 5 or SEQ ID NO: 7.

The DNA hybridizable to the base sequence represented by SEQ ID NO: 2, SEQ ID NO: 4, SEQ ID NO: 6 or SEQ ID NO: 8 includes DNA containing a base sequence having at least about 70% homology, preferably at least about 80% homology, more preferably at least about 90% homology and most preferably at least about 95% homology, to the base sequence represented by SEQ ID NO: 2, SEQ ID NO: 4, SEQ ID NO: 6 or SEQ ID NO: 8; etc.

The hybridization can be carried out by publicly known methods or by modifications of these methods, for example, according to the method described in Molecular Cloning, 2nd (J. Sambrook et al., Cold Spring Harbor Lab. Press, 1989), etc. A commercially available library may also be used according to the instructions of the attached manufacturer's protocol. More preferably, the hybridization can be carried out under highly stringent conditions.

The highly stringent conditions used herein are, for example, those in a sodium concentration at about 19 to 40 mM, preferably about 19 to 20 mM at a temperature of about 50 to 70° C., preferably about 60 to 65° C. In particular, hybridization conditions in a sodium concentration of about 19 mM at a temperature of about 65° C. are most preferred.

More specifically, the DNA encoding GFP consisting of the amino acid sequence represented by SEQ ID NO: 1 includes DNA containing tie base sequence represented by SEQ ID NO: 2 (WO 97/42320), etc.; the DNA encoding GFP consisting of the amino acid sequence represented by SEQ ID NO: 3 includes DNA containing the base sequence represented by SEQ ID NO: 4 (WO 96/23810), etc.; the DNA encoding GFP (GFPuv) consisting of the amino acid sequence represented by SEQ ID NO: 5 includes DNA containing the base sequence represented SEQ ID NO: 6, etc.; the DNA encoding GFP (GFPuv) consisting of the amino acid sequence represented by SEQ ID NO: 5 includes DNA containing the base sequence represented SEQ ID NO: 6, etc. (WO 97/26333), etc.; the DNA encoding GFP (EGFP) consisting of the amino acid sequence represented by SEQ ID NO: 7 includes DNA containing the base sequence represented SEQ ID NO: 8, etc. (NCBI Accession No. AAB0572), etc.

For the DNA encoding EGFP consisting of the amino acid sequence represented by SEQ ID NO: 7, there may be employed DNA consisting of the base sequence represented by SEQ ID NO: 8, etc.

For the DNA encoding ECFP consisting of the amino acid sequence represented by SEQ ID NO: 22, there may be employed DNA consisting of the base sequence represented by SEQ ID NO: 23, etc.

For the DNA encoding EYFP consisting of the amino acid sequence represented by SEQ ID NO: 24, there may be employed DNA consisting of the base sequence represented by SEQ ID NO: 25, etc.

For the DNA encoding DsRED consisting of the amino acid sequence represented by SEQ ID NO: 26, there may be employed DNA consisting of the base sequence represented by SEQ ID NO: 27, etc.

For the DNA encoding EBFP consisting of the amino acid sequence represented by SEQ ID NO: 28, there may be employed DNA consisting of the base sequence represented by SEQ ID NO: 29, etc.

The fusion protein of an orphan receptor and a fluorescent protein can be manufactured using DNA ligated with DNA encoding the orphan receptor protein and DNA encoding GFP.

The ligated DNA is constructed in frame by ligating the 5′ end of DNA encoding GFP with the 3′ end of the base sequence of DNA encoding the orphan receptor.

Also, DNA encoding an amino acid sequence composed of approximately 1 to 5 amino acid residues with a small molecular weight such as Ala, Gly, Ser, etc., which is called a linker, may be inserted between the both DNAs.

The expression vector for the fusion protein of an orphan receptor and a fluorescent protein (hereinafter sometimes simply referred to as the fusion protein) can be manufactured, for example, by (1) preparing a DNA fragment encoding the fusion protein and (2) ligating the DNA fragment at the downstream of a promoter in an appropriate expression vector.

As the fusion protein of a receptor protein for which a ligand has not been identified and a fluorescent protein, preferably used are, for example, fusion proteins of 102 kinds of receptor proteins for which ligands have not been identified, each receptor protein having the amino acid sequence represented by any one of SEQ ID NO: 30 through SEQ ID NO: 131, and GFP consisting of the amino acid sequence represented by SEQ ID NO: 1. In these amino acid sequences represented by any of SEQ ID NO: 30 through SEQ ID NO: 131, the N-terminal methionine residue is deleted in the amino acid sequence represented by SEQ ID NO: 1 and as the case may be, the following alanine residue is further replaced by a threonine residue or a serine residue.

The DNA encoding this fusion protein can be prepared by ligating DNA (SEQ ID NO: 2) encoding GFP consisting of the amino acid sequence represented by SEQ ID NO: 1 at the downstream of the region encoding each of the 102 kinds of receptor proteins for which ligands have not been identified. Specifically, a DNA having the base sequence represented by either one of SEQ ID NO: 132 through SEQ ID NO: 233 is used. In these base sequences represented by any of SEQ ID NO: 132 through SEQ ID NO: 233, the 5′ end methionine residue codon is deleted in the base sequence represented by SEQ ID NO: 2 and as the case may be, the following alanine residue codon is further replaced by a threonine residue codon or a serine residue codon.

Examples of the expression vector include plasmids derived form Escherichia coli (e.g., pCR4, pCR2.1, pBR322, pBR325, pUC12, pUC13), plasmids derived from Bacillus subtilis (e.g., pUB 110, pTP5, pC194), plasmids derived from yeast (e.g., pSH19, pSH15), bacteriophages such as X phage, etc., animal viruses such as retrovirus, vaccinia virus, baculovirus, etc., as well as pA1-11, pXT1, pRc/CMV, pRc/RSV, pcDNAI/Neo, etc.

The promoter used in the vector may be any promoter if it is suitable to function well with a host used for gene expression. For example, in the case of using animal cells as the host, SRα promoter, SV40 promoter, LTR promoter, CMV promoter, HSV-TK promoter, etc. are used.

Further in the case of expressing in various tissues, there are employed insulin II promoter (pancreas), Glycoprotein-α subunit promoter (pituitary), transthyretin promoter (liver), renin promoter (kidney), PSE promoter (prostate), CD2 promoter (T cell), IgG-heavy chain promoter (B cell), scavenger-receptor A promoter (macrophage), etc.

Where the host is bacteria of the genus Escherichia, preferred examples of the promoter include trp promoter, lac promoter, recA promoter, λP_{L} promoter, 1pp promoter, etc. Where the host is bacteria of the genus Bacillus as the host, preferred example of the promoter are SPO1 promoter, SPO2 promoter, penP promoter, etc. When yeast is used as the host, preferred examples of the promoter are PHO5 promoter, PGK promoter, GAP promoter, ADH promoter, etc. When insect cells are used as the host, preferred examples of the promoter include polyhedrin prompter, P10 promoter, etc.

In addition to the foregoing examples, the expression vector may further optionally contain an enhancer, a splicing signal, a polyA addition signal, a selection marker, SV40 replication origin (hereinafter sometimes abbreviated as SV40ori) etc. Examples of the selection marker include dihydrofolate reductase (hereinafter sometimes abbreviated as dhfr) gene [methotrexate (MTX) resistance], ampicillin resistant gene (hereinafter sometimes abbreviated as Amp^{r}), neomycin resistant gene (hereinafter sometimes abbreviated as Neo^{r}, G418 resistance), etc. In particular, when dhfr gene is used as the selection marker in CHO (dhfr⁻) cells, selection can also be made on thymidine free media.

If necessary, a signal sequence that matches with a host is added to the N-terminus of the receptor protein of the present invention. Examples of the signal sequence that can be used are Pho A signal sequence, OmpA signal sequence, etc. in the case of using bacteria of the genus Escherichia as the host; α-amylase signal sequence, subtilisin signal sequence, etc. in the case of using bacteria of the genus Bacillus as the host; MFα signal sequence, SUC2 signal sequence, etc. in the case of using yeast as the host; and insulin signal sequence, α-interferon signal sequence, antibody molecule signal sequence, etc. in the case of using animal cells as the host, respectively.

Using the expression vector containing the DNA encoding the fusion protein of the receptor protein and the fluorescent protein thus constructed, transformants can be manufactured.

Examples of the host, which may be used, are bacteria belonging to the genus Escherichia, bacteria belonging to the genus Bacillus, yeast, insect cells, insects, animal cells, etc.

Specific examples of the bacteria belonging to the genus Escherichia include Escherichia coli K12 DH1 (Proc. Natl. Acad. Sci. U.S.A., 60, 160 (1968)), JM103 (Nucleic Acids Research, 9, 309 (1981)), JA221 (Journal of Molecular Biology, 120, 517 (1978)), HB101 (Journal of Molecular Biology, 41, 459 (1969)), C600 (Genetics, 39, 440 (1954)), DH5α (Inoue, H., Nojima, H., Gene, 96, 23-28 (1-990)), DH10B (Proc. Natl. Acad. Sci. USA, 87,4645-4649 (1990)), etc.

Examples of the bacteria belonging to the genus Bacillus include Bacillus subtilis MI114 (Gene, 24, 255 (1983)), 207-21 (Journal of Biochemistry, 95, 87 (1984)), etc.

Examples of yeast include Saccharomyces cereviseae AH22, AH22R⁻, NA87-11A, DKD-5D, 20B-12, Schizosaccharomyces pombe NCYC1913, NCYC2036, Pichia pastoris, etc.

Examples of insect cells include, for the virus AcNPV, Spodoptera frugiperda cells (Sf cells), MG1 cells derived from mid-intestine of Trichoplusia ni, High Five™ cells derived from egg of Trichoplusia ni, cells derived from Mamestra brassicae, cells derived from Estigmena acrea, etc. Where the virus is BmNPV, Bombyx mori N cells (BmN cells), etc. are used. Examples of the Sf cell which can be used are Sf9 cells (ATCC CRL1711), Sf21 cells (both cells are described in Vaughn, J. L. et al., In Vivo, 13, 213-217 (1977), etc.

As the insect, for example, a larva of Bombyx mori is used (Maeda, et al., Nature, 315, 592 (1985)).

Examples of animal cells, which are used, are monkey cells COS-7, Vero, Chinese hamster cells CHO (hereinafter referred to as CHO cells), dhfr gene deficient Chinese hamster cells CHO (hereinafter simply referred to as CHO (dhfr⁻) cell), mouse L cells, mouse AtT-20, mouse myeloma cells, rat GH3, human FL cells, pancreas-derived cells (RINm5F, HIT-T15, etc.), pituitary-derived cells (GH3, GH1, RC4/BC, etc.), placenta-derived cells (BeWo, JAR, JEG-3, etc.), liver-derived cells (HepG2, etc.), kidney-derived cells (ACHN, etc.), hematocyte-derived cells (H9, THP-1, U-937, etc.), HeLa cells, etc.

Bacteria belonging to the genus Escherichia can be transformed, for example, by the method described in Proc. Natl. Acad. Sci. U.S.A., 69, 2110 (1972), Gene, 17, 107 (1982) or the like.

Bacteria belonging to the genus Bacillus can be transformed, for example, by the method described in Molecular & General Genetics, 168, 111 (1979), etc.

Yeast can be transformed, for example, by the method described in Methods in Enzymology, 194, 182-187 (1991), Proc. Natl. Acad. Sci. U.S.A., 75, 1929 (1978), etc.

Insect cells or insects can be transformed, for example, according to the method described in Bio/Technology, 6, 47-55(1988), etc.

Animal cells can be transformed, for example, according to the method described in Saibo Kogaku (Cell Engineering), extra issue 8, Shin Saibo Kogaku Jikken Protocol (New Cell Engineering Experimental Protocol), 263-267 (1995), published by Shujunsha, or Virology, 52, 456 (1973).

Thus, the transformant transformed with the expression vector containing the DNA encoding the fusion protein described above can be obtained.

Where the host is bacteria belonging to the genus Escherichia or the genus Bacillus, the transformant can be appropriately incubated in a liquid medium which contains materials required for growth of the transformant such as carbon sources, nitrogen sources, inorganic materials, and so on. Examples of the carbon sources include glucose, dextrin, soluble starch, sucrose, etc. Examples of the nitrogen sources include inorganic or organic materials such as ammonium salts, nitrate salts, corn steep liquor, peptone, casein, meat extract, soybean cake, potato extract, etc. Examples of the inorganic materials are calcium chloride, sodium dihydrogenphosphate, magnesium chloride, etc. In addition, yeast extract, vitamins, growth promoting factors etc. may also be added to the medium. Preferably, pH of the medium is adjusted to about 5 to about 8.

A preferred example of the medium for culturing the bacteria belonging to the genus Escherichia is M9 medium supplemented with glucose and Casamino acids [Miller, Journal of Experiments in Molecular Genetics, 431-433, Cold Spring Harbor Laboratory, New York (1972)]. If necessary, a chemical such as 3β-indolylacrylic acid can be added to the medium thereby to activate the promoter efficiently.

Where the bacteria belonging to the genus Escherichia are used as the host, the transformant is usually cultured at about 15° C. to about 43° C. for about 3 hours to about 24 hours. If necessary, the culture may be aerated or stirred.

Where the bacteria belonging to the genus Bacillus are used as the host, the transformant is cultured generally at about 30° C. to about 40° C. for about 6 hours to about 24 hours. If necessary, the culture can be aerated or stirred.

Where yeast is used as the host, the transformant is cultured, for example, in Burkholder's minimal medium [Bostian, K. L. et al., Proc. Natl. Acad. Sci. U.S.A., 77, 4505 (1980)] or in SD medium supplemented with 0.5% Casamino acids [Bitter, G. A. et al., Proc. Nat]. Acad. Sci. U.S.A., 81, 5330 (1984)]. Preferably, pH of the medium is adjusted to about 5 to about 8. In general, the transformant is cultured at about 20° C. to about 35° C. for about 24 hours to about 72 hours. If necessary, the culture can be aerated or stirred.

Where insect cells or insects are used as the host, the transformant is cultured in, for example, Grace's Insect Medium (Grace, T. C. C., Nature, 195, 788 (1962)) to which an appropriate additive such as immobilized 10% bovine serum is added. Preferably, pH of the medium is adjusted to about 6.2 to about 6.4. Normally, the transformant is cultured at about 27° C. for about 3 days to about 5 days and, if necessary, the culture can be aerated or stirred.

Where animal cells are employed as the host, the transformant is cultured in, for example, MEM medium containing about 5% to about 20% fetal calf serum [Science, 122, 501 (1952)), DMEM medium (Virology, 8, 396 (1959)], RPMI 1640 medium [The Journal of the American Medical Association, 199, 519 (1967)], 199 medium [Proceeding of the Society for the Biological Medicine, 73, 1 (1950)], etc. Preferably, pH of the medium is adjusted to about 6 to about 8. The transformant is usually cultured at about 30° C. to about 40° C. for about 15 hours to about 60 hours and, if necessary, the culture can be aerated or stirred.

Suitable media, conditions for culturing, etc. on the respective hosts are publicly known (especially see WO 00/14227, page 24, line 24 to page 26, line 8, EP1111047A2, paragraphs [0090] to [0096]).

As above, the fusion protein can be expressed in the cell membrane of the transformant.

The fusion protein can be separated and purified from the culture described above, for example, by the following procedures.

When the fusion protein is extracted from the culture or cells, after culturing the transformants or cells are collected by a publicly known method and suspended in an appropriate buffer. The transformants or cells are then disrupted by publicly known methods such as ultrasonication, a treatment with lysozyme and/or freeze-thaw cycling, followed by centrifugation, filtration, etc. Thus, the crude extract of the fusion protein can be obtained. The buffer used for the procedures may contain a protein modifier such as urea or guanidine hydrochloride, or a surfactant such as Triton X-100™, etc. When the receptor protein is secreted in the culture, after completion of the cultivation the supernatant can be separated from the transformants or cells to collect the supernatant by a publicly known method.

The fusion protein contained in the supernatant or the extract thus obtained can be purified by appropriately combining the publicly known methods for separation and purification. Such publicly known methods for separation and purification include a method utilizing difference in solubility such as salting out, solvent precipitation, etc.; a method utilizing mainly difference in molecular weight such as dialysis, ultrafiltration, gel filtration, SDS-polyacrylamide gel electrophoresis, etc.; a method utilizing difference in electric charge such as ion exchange chromatography, etc.; a method utilizing difference in specific affinity such as affinity chromatography, etc.; a method utilizing difference in hydrophobicity such as reverse phase high performance liquid chromatography, etc.; a method utilizing difference in isoelectric point such as isoelectrofocusing electrophoresis; and the like.

When the fusion protein thus obtained is in a free form, it can be converted into the salt by publicly known methods or modifications thereof. On the other hand, when the fusion protein is obtained in the form of a salt, it can be converted into the free form or in the form of a different salt by publicly known methods or modifications thereof.

The fusion protein produced by the recombinant can be treated, prior to or after the purification, with an appropriate protein modifying enzyme so that the fusion protein can be appropriately modified to partially remove a polypeptide. Examples of the protein-modifying enzyme include trypsin, chymotrypsin, arginyl endopeptidase, protein kinase, glycosidase or the like.

The activity of the thus produced fusion protein or salts thereof can be determined by a test binding to a labeled ligand, by an enzyme immunoassay using a specific antibody, or the like.

Examples of test compounds which are used for the ligand determination method of the present invention include publicly known ligands (for example, angiotensin, bombesin, canavinoid, cholecystokinin, glutamine, serotonin, melatonin, neuropeptide Y, opioids, purines, vasopressin, oxytocin, PACAP (e.g., PACAP27, PACAP38), secretin, glucagon, calcitonin, adrenomedulin, somatostatin, GHRH, CRF, ACTH, GRP, PTH, VIP (vasoactive intestinal and related polypeptide), somatostatin, dopamine, motilin, amylin, bradykinin, CGRP (calcitonin gene-related peptide), leukotriens, pancreastatin, prostaglandins, thromboxane, adenosine, adrenaline, the chemokine superfamily (e.g., the CXC chemokine subfamily such as IL-8, GROα, GROβ, GROγ, NAP-2, ENA-78, GCP-2, PF4, IP-10, Mig, PBSF/SDF-1, etc.; the CC chemokine subfamily such as MCAF/MCP-1, MCP-2, MCP-3, MCP-4, eotaxin, RANTES, MIP-1α, MIP-1β, HCC-1, MIP-3α/LARC, MIP-3β/ELC, I-309, TARC, MIPF-1, MIPF-2/eotaxin-2, MDC, DC-CK1/PARC, SLC, etc.; the C chemokine subfamily such as lymphotactin, etc.; the CX3C chemokine subfamily such as fractalkine, etc.), endothelin, enterogastrin, histamine, neurotensin, TRH, pancreatic polypeptide, galanin, lysophosphatidic acid (LPA), sphingosine 1-phosphate, lysophosphatidylserine, sphingosylphosphorylcholine, lyzophosphatidylcholine, steroids, bile acids, isoprenoids, arachidonic acid metabolites, amines, amino acids, nucleotides, nucleosides, saturated or saturated fatty acids, etc.) as well as other substances, for example, tissue extracts, cell culture supernatants from humans or mammals (e.g., mice, rats, swine, bovine, sheep, monkeys, etc.). For example, the tissue extract, cell culture supernatant, etc. is added to the cell wherein the fusion protein of the orphan receptor and GFP has been expressed, and screening is made while assaying the cell-stimulating activities, etc. to finally determine a single ligand.

Specifically, the ligand determination method A of the present invention is a method which involves constructing the fusion protein-expressed cell and assaying cell stimulating activities or perform a receptor binding assay by using the expressed cell or its cell membrane fraction, thus determining compounds that bind to the orphan receptors to have the cell stimulating activities (e.g., activities that accelerate or suppress arachidonic acid release, acetylcholine release, intracellular Ca²⁺ release, intracellular cAMP production, intracellular cGMP production, inositol phosphate production, changes in cell membrane potential, phosphorylation of intracellular proteins, activation of c-fos, pH reduction, etc.), MAP kinase activation, activation of transcription factors (e.g., CRE, AP1, NFκB, etc.), diacylglycerol production, opening or closing of ion channels (e.g., K⁺, Ca²⁺, Na⁺, Cl⁻, etc.) on cell membranes, apoptosis induction, morphological changes in cells, transport of the receptor (fusion protein) from cell membrane to cytoplasm, activation of low molecular weight G proteins (e.g., Ras, Rap, Rho, Rab, etc.), cell division-promoting activity, DNA synthesis-promoting activity, etc., that is, ligands (e.g., peptides, proteins, non-peptide compounds, synthetic compounds, fermentation products, etc.).

The ligand determination methods of the present invention are characterized by assaying, e.g., the binding amount of a test compound to the orphan receptor or the cell stimulating activities, etc., when a cell wherein the fusion protein has been expressed or a membrane fraction of the cell is brought in contact with the test compound.

More specifically, the present invention provides the following ligand determination methods:
(1) a method of determining a ligand to the orphan receptor, which comprises assaying the amount of a labeled test compound bound to a cell wherein the fusion protein has been expressed, when the labeled test compound is brought in contact with the cell or the cell membrane fraction;
(2) a method of determining a ligand to the orphan receptor, which comprises assaying the amount of a labeled test compound bound to a cell wherein the fusion protein has been expressed on a cell membrane by culturing a transformant containing DNA encoding the fusion protein, when the labeled test compound is brought in contact with the fusion protein;
(3) a method of determining a ligand to the orphan receptor, which comprises assaying the aforesaid cell stimulating activities, MAP kinase activation, activation of transcription factors (e.g., CRE, AP1, NFκB, etc.), diacylglycerol production, opening or closing of ion channels (e.g., K⁺, Ca²⁺, Na⁺, Cl⁻, etc.) on cell membranes, apoptosis induction, morphological changes in cells, transport of the receptor (fusion protein) from cell membrane to cytoplasm, activation of low molecular weight G proteins (e.g., Ras, Rap, Rho, Rab, etc.), cell division-promoting activity, DNA synthesis-promoting activity, etc., which are mediated by the orphan receptor, when a test compound is brought in contact with a cell wherein the fusion protein has been expressed; and,
(4) a method of determining a ligand to the orphan receptor, which comprises assaying the aforesaid mediated the cell stimulating activities, MAP kinase activation, activation of transcription factors (e.g., CRE, AP1, NFκB, etc.), diacylglycerol production, opening or closing of ion channels (e.g., K⁺, Ca²⁺, Na⁺, Cl⁻, etc.) on cell membranes, apoptosis induction, morphological changes in cells, transport of the receptor (fusion protein) from cell membrane to cytoplasm, activation of low molecular weight G proteins (e.g., Ras, Rap, Rho, Rab, etc.), cell division-promoting activity, DNA synthesis-promoting activity, etc., which are mediated by the orphan receptor, when a test compound is brought in contact with the fusion protein expressed on a cell membrane by culturing a transformant containing DNA encoding the fusion protein.

In particular, it is preferred to perform the tests (1) and (2) described above, thereby to confirm that the test compound can bind to the orphan receptor, followed by the tests (3) and (4) described above.

As the case may be, the fusion protein can be isolated and purified from the expression cell described above and the receptor binding assay, etc. may be carried out using the same. For the fusion protein used in the ligand determination method, fusion proteins abundantly expressed using the cells described above are appropriate.

The fusion protein can be manufactured by the method for expression described above, preferably by expressing DNA encoding the fusion protein in mammalian or insect cells, etc. For introducing a DNA fragment encoding the fusion protein into host animal cells and efficiently expressing the same, it is preferred to insert the DNA fragment downstream a polyhedrin promoter of nuclear polyhedrosis virus (NPV) belonging to a baculovirus, an SV40-derived promoter, a retrovirus promoter, a metallothionein promoter, a human heat shock promoter, a cytomegalovirus promoter, an SRa promoter or the like. The amount and quality of the fusion protein expressed can be determined by a publicly known method. For example, this determination can be made by the method described in the literature [Nambi, P., et al., J. Biol. Chem., 267, 19555-19559 (1992)].

In the ligand determination methods of the present invention, it is preferred to use the fusion protein-expressed cell or the cell membrane fraction.

Where the fusion protein-expressed cells are used in the ligand determination methods of the present invention, the cells may be fixed using glutaraldehyde, formalin, etc. The fixation can be made by publicly known methods.

The cells wherein the fusion protein has been expressed refer to host cells wherein the fusion protein has been expressed, which host cells include Escherichia coli, Bacillus subtilis, yeast, insect cells, animal cells, and the like.

The cell membrane fraction means a fraction abundant in cell membrane obtained by cell disruption and subsequent fractionation by publicly known methods. Useful cell disruption methods include cell squashing using a Potter-Elvehjem homogenizer, disruption using a Waring blender or Polytron (manufactured by Kinematica Inc.), disruption by ultrasonication, disruption by cell spraying through thin nozzles under an increased pressure using a French press, or the like. Cell membrane fractionation is effected mainly by fractionation using a centrifugal force, such as centrifugation for fractionation and density gradient centrifugation. For example, cell disruption fluid is centrifuged at a low speed (500 rpm to 3,000 rpm) for a short period of time (normally about 1 to about 10 minutes), the resulting supernatant is then centrifuged at a higher speed (15,000 rpm to 30,000 rpm) normally for 30 minutes to 2 hours. The precipitate thus obtained is used as the membrane fraction. The membrane fraction is rich in the fusion protein expressed and membrane components such as cell-derived phospholipids, membrane proteins, etc.

The amount of the fusion protein in the fusion protein-expressed cells or the cell membrane fraction is preferably 10³ to 10⁸ molecules per cell, more preferably 10⁵ to 10⁷ molecules per cell. As the amount of expression increases, the ligand binding activity per unit of membrane fraction (specific activity) increases so that not only the highly sensitive screening system can be constructed but also large quantities of samples can be assayed with the same lot. The amount of this fusion protein expressed can be roughly estimated from the fluorescence level of GFP in the cells or cell membranes, using a fluorescence microscope or a fluorophotometer.

To perform the methods (1) and (2) described above for determining a ligand to the orphan receptor, appropriate cells or cell membrane fractions containing the fusion protein and a labeled test compound are required. The fusion protein fraction is desirably a fraction containing a recombinant fusion receptor having an activity equivalent to a naturally occurring fusion protein. Herein, the term “oequivalent activity” means a ligand binding activity, a signal transduction activity, etc., which is equivalent.

As the labeled test compounds, there are used compounds selected from the aforesaid group of ligand compounds, which are labeled with [³H], [¹²⁵I], [¹⁴C], [³⁵S], etc.

Specifically, the ligand determination methods of the present invention can be performed by the following procedures. First, a standard fusion protein preparation is prepared by suspending cells wherein the fusion protein has been expressed or the cell membrane fraction in a buffer appropriate for use in the determination method. Any buffer can be used so long as it does not inhibit the ligand-receptor binding, such buffers including a phosphate buffer or a Tris-HCl buffer having pH of 4 to 10 (preferably pH of 6 to 8). For the purpose of minimizing non-specific binding, a surfactant such as CHAPS, Tween-80™ (manufactured by Kao-Atlas Inc.), digitonin or deoxycholate, and various proteins such as bovine serum albumin or gelatin, may optionally be added to the buffer. Further for the purpose of suppressing the degradation of the receptors or ligands by proteases, a protease inhibitor such as PMSF, leupeptin, E-64 (manufactured by Peptide Institute, Inc.) and pepstatin may also be added. A given amount (5,000 to 500,000 cpm) of the test compound labeled with [³H], [¹²⁵I], [¹⁴C], [³⁵S] or the like is added to 0.01 ml to 10 ml of the receptor preparation. To determine the amount of non-specific binding (NSB), a reaction tube containing an unlabeled test compound in a large excess is also prepared. The reaction is carried out at approximately 0 to 50° C., preferably about 4 to 37° C. for about 20 minutes to about 24 hours, preferably about 30 minutes to about 3 hours. After completion of the reaction, the reaction mixture is filtrated through glass fiber filter paper, etc. and washed with an appropriate volume of the same buffer. The residual radioactivity on the glass fiber filter paper is then measured by means of a liquid scintillation counter or γ-counter. A test compound exceeding 0 cpm in count obtained by subtracting nonspecific binding (NSB) from the total binding (B) (B minus NSB) may be selected as a ligand (including an agonist) to the orphan receptor.

The method (3) or (4) of the present invention described above for determining a ligand can be performed as follows. The aforesaid orphan receptor-mediated cell stimulating activities, for example, MAP kinase activation, activation of transcription factors (e.g., CRE, AP1, NFκB, etc.), diacylglycerol production, opening or closing of ion channels (e.g., K⁺, Ca²⁺, Na⁺, Cl⁻, etc.) on cell membranes, apoptosis induction, morphological changes in cells, transport of the receptor (fusion protein) from cell membrane into cytoplasm, activation of low molecular weight G proteins (e.g., Ras, Rap, Rho, Rab, etc.), cell division-promoting activity, DNA synthesis-promoting activity, etc. can be assayed by publicly known methods or using assay kits commercially available. Specifically, cells wherein the fusion protein has expressed are first cultured on a multi-well plate, etc. Prior to the ligand determination, the medium is replaced with fresh medium or with an appropriate non-cytotoxic buffer, followed by incubation for a given period of time in the presence of a test compound, etc. Subsequently, the cells are extracted or the supernatant is recovered and the resulting product is quantified by appropriate procedures. Where it is difficult to detect the production of the indicator substance (e.g., arachidonic acid, etc.) for the cell-stimulating activity due to a degrading enzyme contained in the cells, an inhibitor against such a degrading enzyme may be added prior to the assay. For detecting activities such as the cAMP production suppression activity, the baseline production in the cells is increased by forskolin or the like and the suppressing effect on the increased baseline production may then be detected.

In the cell stimulating activities described above, “transport of the receptor to cytoplasm” is assayed by measuring the fluorescence of a fluorescent protein such as GFP, etc., whereby movement of the fusion protein from the cell membrane to the cytoplasm can be observed. In order to observe the movement of the fusion protein from the cell membrane to the cytoplasm, it is suited to use animal cells which have expressed the aforesaid fusion protein stably or transiently. The cells are incubated in an appropriate incubator using an ordinary medium and a test compound diluted to a suitable concentration is added to the cells. In this case, a diluted solution of the test compound may be added directly to the medium, or after the cells are washed with Hanks' balanced salt solution (HBSS, to which BSA may be added in 0 to 10%, preferably 0.1 to 1%), HBSS containing the test compound may be added to the cells. The cells are allowed to stand at 4° C. to 37° C., preferably at 20° C. to 37° C. for 1 minute to 6 hours, preferably for 10 minutes to 2 hours, followed by observing movement of the fusion protein from the cell membrane to the cytoplasm. Though the cells can be observed as they are, the cells may also be fixed with glutaraldehyde, formalin, etc. The cells can be fixed by publicly known methods.

Observation may be made using an ordinary fluorescence microscope or confocal laser scanning microscope. Also, a plate reader having the function to irradiate excitement light and the function to take fluorescent images in can be used. In this case, after excitement with ultraviolet light, preferably at 395 nm, the fusion protein of the receptor and wild type GFP or GFPuv represented by SEQ ID NO: 3 or SEQ ID NO: 5 can be detected using fluorescein isothianate (FITC) or filters normally commercially available for detecting GFP. In order to detect the fusion protein with GFP represented by SEQ ID NO: 1 or SEQ ID NO: 7, excitement may be effected with excitement light at 460-500 nm, preferably at 488 nm and observation may be made using FITC or filters normally commercially available for detecting GFP.

In addition to the transport of the receptor (fusion protein) into cytoplasm, morphological changes of the receptor including receptor aggregation, receptor localization, decreased or increased expression of the receptor, etc., which can be microscopically observed, may also be observed.

In practicing the ligand determination method B of the present invention, it is necessary to incorporate a plasmid containing a DNA encoding the reporter protein at the downstream of a particular enhancer/promoter into cells, preferably eukaryote-derived cells, in addition to the expression vector for the fusion protein. This plasmid may contain a promoter capable of expressing the reporter protein in cells, preferably in eukaryote-derived cells, may further contain a chemical resistant gene (e.g., ampicillin-resistant gene), etc. as a selection marker in the case of proliferating in eukaryotes.

The plasmid containing a DNA encoding the reporter protein at the downstream of the enhancer/promoter may be any plasmid such as commercially available plasmid, etc., so far as it is capable of expressing the reporter protein in cells under control of the enhancer and is capable of introducing into cells.

As the enhancer, there is used, for example, a virus-derived enhancer such as papilloma virus, etc., LTR of a retrovirus, cAMP response element (CRE), TPA response element (TRE) or the like, preferably cAMP response element. An enhancer which can be activated by the aforesaid cell stimulating activities mediated by the orphan receptor the cells express is appropriate.

As the promoter, there is used, for example, SV40 promoter, a TATA-like promoter of HSV thymidine kinase gene, etc., with a TATA-like promoter being preferred.

As the reporter protein gene, there is used, for example, luciferase gene, β-galactosidase gene, GFP gene, alkaline phosphatase gene, etc. Any enzyme gene is usable as the reporter gene so long as its enzyme activity can be detected by publicly known methods.

Specific examples of such plasmids include a plasmid ligated with a TATA-like promoter and a reporter protein (e.g., luciferase gene) at the downstream of cAMP response element, e.g., pCRE-Luc (Clontech, Inc.), etc.

The cells used in the ligand determination method B are the host cells described above, preferably animal cells (e.g., monkey cells COS-7, Vero, CHO cells, CHO (dhfr⁻) cells, mouse L cells, mouse AtT-20, mouse myeloma cells, rat GH3, human FL cells, human HEK293 cells, etc.) and the like.

The cells may express 2 or more (preferably 2 or 3) fusion proteins.

Where 2 or more fusion proteins are expressed, it is preferred to use 2 or more orphan receptors having similar biological characteristics.

Examples of the similar biological characteristics include expression levels of the reporter protein when 2 or more orphan receptors used are expressed independently; etc. Specifically, the characteristics of the respective receptor proteins can be distinguished, using as an indicator (1) the basic expression level of the reporter protein and/or (2) the expression level of the reporter protein in the presence of forskolin, when 2 or more orphan receptors are expressed individually.

Accordingly, when 2 or more orphan receptors are expressed to determine the ligand, it is desired to previously sort the reporter proteins into those having low, medium and obviously high basic expression levels, etc., or to clarify the receptor protein difficult to increase an expression level of the reporter protein by the addition of forskolin. This is because where two receptor protein, for example, a receptor protein having a high basic expression level of the reporter protein and a receptor having a low basic expression level of the reporter protein are expressed, it becomes difficult to detect an increase in the expression level of the reporter protein when a ligand binds to the latter. That is:
(1) it is preferred to avoid the mixture of an orphan receptor that the basic expression level of the reporter protein is high and a receptor protein that the basic expression level is low;
(2) it is preferred not to mix a receptor protein that the expression level of the reporter protein increased by the addition of forskolin is remarkably high with an orphan receptor that the expression level increased is not; and,
(3) it is preferred that orphan receptors having almost equal basic expression levels of the reporter protein are used and expressed in combination.

As such, the combination of orphan receptors having similar characteristics includes, for example, the combination of APJ (apelin receptor; Gene, 136, 355 (1993)) and TGR-1 (Japanese Patent Application KOKAI No. 2002-078492).

The ligand determination method B is specifically described below.

Cells are plated on a 96-well plate and incubated overnight in DMEM containing, e.g., 10% fetal calf serum. At this stage, an expression plasmid and a reporter plasmid in the fluorescent protein are co-transfected to the cells, using a transfection kit commercially available. The cells are further incubated overnight to express the orphan receptor transiently in the cells. After the cells are washed and the medium is made serum-free, a test compound is added. Where the enhancer is CRE, forskolin may be added simultaneously with the test compound. After incubation is carried out for a given period of time, the cells are lysed and the activities of the reporter protein are assayed.

In the determination method described above, when the base line for the activities of the reporter protein is high so that a change in the activities is detected by a test compound only with difficulties, it is preferred to take measures for reducing the base line. For example in the case that the orphan receptor is a G protein-coupled receptor protein (GPCR), Gi protein, among a subunits of G protein, showing a cAMP inhibitory effect is added, which makes the detection of a change in activities easy. To express Gi protein, a plasmid capable of expressing a DNA encoding Gi protein can be transfected to cells together with the orphan receptor plasmid and the reporter plasmid. In this case, a mixing ratio of these three plasmids (the orphan receptor plasmid, the reporter plasmid and the Gi plasmid) are preferably in about 5-15:1:1 to 6, more preferably in about 7:1:3.

In the ligand determination method B of the present invention, where the activities of the reporter protein increase or decrease by about 20% or more, preferably by about 50% or more when a test compound is added, the test compound can be identified to be a ligand.

The test compound used in the ligand determination method B of the present invention is a compound selected from the test compounds described above.

Furthermore, the kit of the present invention for determination of the ligand contains cells capable of expressing the fusion protein of the present invention or its cell membrane fraction, etc.

Examples of the ligand determination kit of the present invention are given below.

1. Reagents for Determining Ligands

(1) Assay Buffer and Wash Buffer

Hanks' Balanced Salt Solution (manufactured by Gibco Co.) supplemented with 0.05% bovine serum albumin (Sigma Co.).

The solution is sterilized by filtration through a 0.45 μm filter and stored at 4° C. Alternatively, the solution may be prepared at use.

(2) Fusion Protein Preparation

CHO cells wherein the fusion protein has been expressed are passaged in a 12-well plate in a density of 5×10⁵ cells/well followed by culturing at 37° C. under 5% CO₂ and 95% air for 2 days.

(3) Labeled Test Compound

Compounds labeled with [³H], [¹²⁵I], [¹⁴C], [³⁵S], etc., which are commercially available labels, or compounds labeled by appropriate methods.

An aqueous solution of the compound is stored at 4° C. or −20° C. The solution is diluted to 1 μM with an assay buffer at use. A sparingly water-soluble test compound is dissolved in dimethylformamide, DMSO, methanol, etc.

(4) Non-Labeled Test Compound

A non-labeled form of the same compound as the labeled compound is prepared in a concentration of 100 to 1,000-fold higher than that of the labeled compound.

2. Assay Method

(1) CHO cells wherein the fusion protein has been expressed are cultured in a 12-well culture plate. After washing twice with 1 ml of an assay buffer, 490 μl of the assay buffer is added to each well.
(2) After 5 μl of the labeled test compound is added, the resulting mixture is incubated at room temperature for an hour. To determine the non-specific binding, 5 μl of the non-labeled compound is added to the system.
(3) The reaction mixture is removed and the wells are washed 3 times with 1 ml of washing buffer. The labeled test compound bound to the cells is dissolved in 0.2N NaOH-1% SDS and then mixed with 4 ml of liquid scintillator A (manufactured by Wako Pure Chemical Industries, Ltd.).
(4) The radioactivity is measured using a liquid scintillation counter (manufactured by Beckman Co.).

As such, according to the ligand determination methods (A and B) of the present invention, immunostaining or western blot assay utilizing the fluorescence from the fluorescent protein such as GFP, etc. or a fluorescent protein antibody such as a GFP antibody, etc. is used to effect (1) to (5) below:
(1) it can be confirmed that the receptor protein has been expressed on a protein level;
(2) it can be confirmed that the receptor protein has been expressed on a cell membrane;
(3) an expression level. of the receptor protein can be estimated;
(4) a cell wherein the receptor protein has been expressed abundantly can be selected; and,
(5) a specific reaction of the receptor by a ligand can be detected as internalization of the fusion protein of the receptor and the fluorescent protein into cells. By utilizing these features, a ligand to the receptor protein for which a ligand has not been identified (orphan receptor) can be determined efficiently.

The ligand thus determined binds to its receptor protein to regulate its physiological functions and thus can be used as an agent for the prevention and/or treatment of diseases associated with the functions of the receptor protein. Further using the ligand and its receptor protein, an agonist/antagonist to the receptor can be screened.

In the specification and drawings, the codes of bases and amino acids are denoted in accordance with the IUPAC-IUB Commission on Biochemical Nomenclature or by the common codes in the art, examples of which are shown below. For amino acids that may have the optical isomer, L form is presented unless otherwise indicated.

The sequence identification numbers in the sequence listing of the specification indicates the following sequence, respectively.

[SEQ ID NO: 1]

This shows the amino acid sequence of GFP used in EXAMPLE 1 (hereinafter merely referred to as GFP-1).

SEQ ID NO: 2

This shows the base sequence of DNA encoding GFP used in EXAMPLE 1.

SEQ ID NO: 3

This shows the amino acid sequence of wild type GFP.

SEQ ID NO: 4

This shows the base sequence of DNA encoding wild type GFP.

SEQ ID NO: 5

This shows the amino acid sequence of GFPuv.

SEQ ID NO: 6

This shows the base sequence of DNA encoding GFPuv.

SEQ ID NO: 7

This shows the amino acid sequence of EGFP.

SEQ ID NO: 8

This shows the base sequence of cDNA encoding EGFP.

SEQ ID NO: 9

This shows the amino acid sequence of human-derived G protein-coupled receptor protein TGR5 used in EXAMPLE 1.

SEQ ID NO: 10

This shows the base sequence of cDNA encoding human-derived G protein-coupled receptor protein TGR5 used in EXAMPLE 1.

SEQ ID NO: 11

This shows the base sequence of primer 1 used for PCR in REFERENCE EXAMPLE 1.

SEQ ID NO: 12

This shows the base sequence of primer 2 used for PCR in REFERENCE EXAMPLE 1.

SEQ IDNO: 13

This shows the amino acid sequence of human-derived parathyroid hormone receptor (PTH-R).

SEQ ID NO: 14

This shows the base sequence of cDNA encoding human-derived parathyroid hormone receptor (PTH-R).

SEQ ID NO: 15

This shows the amino acid sequence of human-derived GPR40.

SEQ ID NO: 16

This shows the base sequence of cDNA encoding human-derived GPR40.

SEQ ID NO: 17

This shows the amino acid sequence of His-Tag.

SEQ ID NO: 18

This shows the amino acid sequence of V5-tag.

SEQ ID NO: 19

This shows the amino acid sequence of myc-tag.

SEQ ID NO: 20

This shows the amino acid sequence of Xpress-tag.

SEQ ID NO: 21

This shows the amino acid sequence of HA-tag.

SEQ ID NO: 22

This shows the amino acid sequence of ECFP.

SEQ ID NO: 23

This shows the base sequence of cDNA encoding ECFP.

SEQ ID NO: 24

This shows the amino acid sequence of EYFP.

SEQ ID NO: 25

This shows the base sequence of cDNA encoding EYFP.

SEQ ID NO: 26

This shows the amino acid sequence of DsRED.

SEQ ID NO: 27

This shows the base sequence of cDNA encoding DsRED.

SEQ ID NO: 28

This shows the amino acid sequence of EBFP.

SEQ ID NO: 29

This shows the base sequence of cDNA encoding EBFP.

SEQ ID NO: 30

This shows the amino acid sequence of the fusion protein of orphan receptor hBL5 and GFP-1.

SEQ ID NO: 31

This shows the amino acid sequence of the fusion protein of orphan receptor h7TBA62 and GFP-1.

SEQ ID NO: 32

This shows the amino acid sequence of the fusion protein of orphan receptor 14273 and GFP-1.

SEQ ID NO: 33

This shows the amino acid sequence of the fusion protein of orphan receptor EMR3 and GFP-1.

SEQ ID NO: 34

This shows the amino acid sequence of the fusion protein of orphan receptor GPR15 and GFP-1.

SEQ ID NO: 35

This shows the amino acid sequence of the fusion protein of orphan receptor GPR31 and GFP-1.

SEQ ID NO: 36

This shows the amino acid sequence of the fusion protein of orphan receptor GPRC5B and GFP-1.

SEQ ID NO: 37

This shows the amino acid sequence of the fusion protein of orphan receptor PSEC0142 and GFP-1.

SEQ ID NO: 38

This shows the amino acid sequence of the fusion protein of orphan receptor HE6 and GFP-1.

SEQ ID NO: 39

This shows the amino acid sequence of the fusion protein of orphan receptor GPR61 and GFP-1.

SEQ ID NO: 40

This shows the amino acid sequence of the fusion protein of orphan receptor TGR9 and GFP-1.

SEQ ID NO: 41

This shows the amino acid sequence of the fusion protein of orphan receptor TGR24 and GFP-1.

SEQ ID NO: 42

This shows the amino acid sequence of the fusion protein of orphan receptor ZGPR1 and GFP-1.

SEQ ID NO: 43

This shows the amino acid sequence of the fusion protein of orphan receptor EMR1 and GFP-1.

SEQ ID NO: 44

This shows the amino acid sequence of the fusion protein of orphan receptor GPR25 and GFP-1.

SEQ ID NO: 45

This shows the amino acid sequence of the fusion protein of orphan receptor GPR55 and GFP-1.

SEQ ID NO: 46

This shows the amino acid sequence of the fusion protein of orphan receptor AXOR14 and GFP-1.

SEQ ID NO: 47

This shows the amino acid sequence of the fusion protein of orphan receptor TM7SF1 and GFP-1.

SEQ ID NO: 48

This shows the amino acid sequence of the fusion protein of orphan receptor PSP24B and GFP-1.

SEQ ID NO: 49

This shows the amino acid sequence of the fusion protein of orphan receptor SREB3 and GFP-1.

SEQ ID NO: 50

This shows the amino acid sequence of the fusion protein of orphan receptor TGR37 and GFP-1.

SEQ ID NO: 51

This shows the amino acid sequence of the fusion protein of orphan receptor H963 and GFP-1.

SEQ ID NO: 52

This shows the amino acid sequence of the fusion protein of orphan receptor GPR87 and GFP-1.

SEQ ID NO: 53

This shows the amino acid sequence of the fusion protein of orphan receptor GPR91 and GFP-1.

SEQ ID NO: 54

This shows the amino acid sequence of the fusion protein of orphan receptor PNR and GFP-1.

SEQ ID NO: 55

This shows the amino acid sequence of the fusion protein of orphan receptor TGR29 and GFP-1.

SEQ ID NO: 56

This shows the amino acid sequence of the fusion protein of orphan receptor TGR36 and GFP-1.

SEQ ID NO: 57

This shows the amino acid sequence of the fusion protein of orphan receptor H9 and GFP-1.

SEQ ID NO: 58

This shows the amino acid sequence of the fusion protein of orphan receptor TGR18 and GFP-1.

SEQ ID NO: 59

This shows the amino acid sequence of the fusion protein of orphan receptor TGR19 and GFP-1.

SEQ ID NO: 60

This shows the amino acid sequence of the fusion protein of orphan receptor AM-R and GFP-1.

SEQ ID NO: 61

This shows the amino acid sequence of the fusion protein of orphan receptor GPRI9 and GFP-1:

SEQ ID NO: 62

This shows the amino acid sequence of the fusion protein of orphan receptor GPR45 and GFP-1.

SEQ ID NO: 63

This shows the amino acid sequence of the fusion protein of orphan receptor GPRC5D and GFP-1.

SEQ ID NO: 64

This shows the amino acid sequence of the fusion protein of orphan receptor LGR6 and GFP-1.

SEQ ID NO: 65

This shows the amino acid sequence of the fusion protein of orphan receptor RUP3 and GFP-1.

SEQ ID NO: 66

This shows the amino acid sequence of the fusion protein of orphan receptor TGR14 and GFP-1.

SEQ ID NO: 67

This shows the amino acid sequence of the fusion protein of orphan receptor TPRA40 and GFP-1.

SEQ ID NO: 68

This shows the amino acid sequence of the fusion protein of orphan receptor GPR22 and GFP-1.

SEQ ID NO: 69

This shows the amino acid sequence of the fusion protein of orphan receptor GPR52 and GFP-1.

SEQ ID NO: 70

This shows the amino acid sequence of the fusion protein of orphan receptor FLH2882 and GFP-1.

SEQ ID NO: 71

This shows the amino acid sequence of the fusion protein of orphan receptor SNORF36 and GFP-1.

SEQ ID NO: 72

This shows the amino acid sequence of the fusion protein of orphan receptor MRG and GFP-1.

SEQ ID NO: 73

This shows the amino acid sequence of the fusion protein of orphan receptor SREB2 and GFP-1.

SEQ ID NO: 74

This shows the amino acid sequence of the fusion protein of orphan receptor GPR12 and GFP-1.

SEQ ID NO: 75

This shows the amino acid sequence of the fusion protein of orphan receptor GPR30 and GFP-1.

SEQ ID NO: 76

This shows the amino acid sequence of the fusion protein of orphan receptor GPR82 and GFP-1.

SEQ ID NO: 77

This shows the amino acid sequence of the fusion protein of orphan receptor RECAP and GFP-1.

SEQ ID NO: 78

This shows the amino acid sequence of the fusion protein of orphan receptor HB954 and GFP-1.

SEQ ID NO: 79

This shows the amino acid sequence of the fusion protein of orphan receptor RDC 1 and GFP-1.

SEQ ID NO: 80

This shows the amino acid sequence of the fusion protein of orphan receptor TGR6 and GFP-1.

SEQ ID NO: 81

This shows the amino acid sequence of the fusion protein of orphan receptor A-2 and GFP-1.

SEQ ID NO: 82

This shows the amino acid sequence of the fusion protein of orphan receptor JEG1 8 and GFP-1.

SEQ ID NO: 83

This shows the amino acid sequence of the fusion protein of orphan receptor GPR17 and GFP-1.

SEQ ID NO: 84

This shows the amino acid sequence of the fusion protein of orphan receptor GPR35 and GFP-1.

SEQ ID NO: 85

This shows the amino acid sequence of the fusion protein of orphan receptor GPRC5C and GFP-1.

SEQ ID NO: 86

This shows the amino acid sequence of the fusion protein of orphan receptor HM74 and GFP-1.

SEQ ID NO: 87

This shows the amino acid sequence of the fusion protein of orphan receptor RPE and GFP-1.

SEQ ID NO: 88

This shows the amino acid sequence of the fusion protein of orphan receptor TGR13 and GFP-1.

SEQ ID NO: 89

This shows the amino acid sequence of the fusion protein of orphan receptor TGR27 and GFP-1.

SEQ ID NO: 90

This shows the amino acid sequence of the fusion protein of orphan receptor DEZ and GFP-1.

SEQ ID NO: 91

This shows the amino acid sequence of the fusion protein of orphan receptor ratGPR1 and GFP-1.

SEQ ID NO: 92

This shows the amino acid sequence of the fusion protein of orphan receptor GPR3 and GFP-1.

SEQ ID NO: 93

This shows the amino acid sequence of the fusion protein of orphan receptor GPR6 and GFP-1.

SEQ ID NO: 94

This shows the amino acid sequence of the fusion protein of orphan receptor RAIG1 and GFP-1.

SEQ ID NO: 95

This shows the amino acid sequence of the fusion protein of orphan receptor TGR2-1 and GFP-1.

SEQ ID NO: 96

This shows the amino acid sequence of the fusion protein of orphan receptor TGR2-2 and GFP-1.

SEQ ID NO: 97

This shows the amino acid sequence of the fusion protein of orphan receptor TGR21 and GFP-1.

SEQ ID NO: 98

This shows the amino acid sequence of the fusion protein of orphan receptor GPR56 and GFP-1.

SEQ ID NO: 99

This shows the amino acid sequence of the fusion protein of orphan receptor KIAA0758 and GFP-1.

SEQ ID NO: 100

This shows the amino acid sequence of the fusion protein of orphan receptor RE2 and GFP-1.

SEQ ID NO: 101

This shows the amino acid sequence of the fusion protein of orphan receptor P40 and GFP-1.

SEQ ID NO: 102

This shows the amino acid sequence of the fusion protein of orphan receptor GPR27 and GFP-1.

SEQ ID NO: 103

This shows the amino acid sequence of the fusion protein of orphan receptor HG38 and GFP-1.

SEQ ID NO: 104

This shows the amino acid sequence of the fusion protein of orphan receptor DRR1 and GFP-1.

SEQ ID NO: 105

This shows the amino acid sequence of the fusion protein of orphan receptor TGR12 and GFP-1.

SEQ ID NO: 106

This shows the amino acid sequence of the fusion protein of orphan receptor TGR11 and GFP-1.

SEQ ID NO: 107

This shows the amino acid sequence of the fusion protein of orphan receptor TGR15 and GFP-1.

SEQ ID NO: 108

This shows the amino acid sequence of the fusion protein of orphan receptor TGR8 and GFP-1.

SEQ ID NO: 109

This shows the amino acid sequence of the fusion protein of orphan receptor GPR20 and GFP-1.

SEQ ID NO: 110

This shows the amino acid sequence of the fusion protein of orphan receptor TGR10 and GFP-1.

SEQ ID NO: 111

This shows the amino acid sequence of the fusion protein of orphan receptor TGR30 and GFP-1.

SEQ ID NO: 112

This shows the amino acid sequence of the fusion protein of orphan receptor GPR18 and GFP-1.

SEQ ID NO: 113

This shows the amino acid sequence of the fusion protein of orphan receptor TGR25 and GFP-1.

SEQ ID NO: 114

This shows the amino acid sequence of the fusion protein of orphan receptor GPR23 and GFP-1.

SEQ ID NO: 115

This shows the amino acid sequence of the fusion protein of orphan receptor P2Y10 and GFP-1.

SEQ ID NO: 116

This shows the amino acid sequence of the fusion protein of orphan receptor GPR37 and GFP-1.

SEQ ID NO: 117

This shows the amino acid sequence of the fusion protein of orphan receptor ET(B)R-LP-2 and GFP-1.

SEQ ID NO: 118

This shows the amino acid sequence of the fusion protein of orphan receptor FPRL2 and GFP-1.

SEQ ID NO: 119

This shows the amino acid sequence of the fusion protein of orphan receptor GPR32 and GFP-1.

SEQ ID NO: 120

This shows the amino acid sequence of the fusion protein of orphan receptor dj287G14.2 and GFP-1.

SEQ ID NO: 121

This shows the amino acid sequence of the fusion protein of orphan receptor BRS-3 and GFP-1.

SEQ ID NO: 122

This shows the amino acid sequence of the fusion protein of orphan receptor GPR39 and GFP-1.

SEQ ID NO: 123

This shows the amino acid sequence of the fusion protein of orphan receptor 63A2 and GFP-1.

SEQ ID NO: 124

This shows the amino acid sequence of the fusion protein of orphan receptor GPR84 and GFP-1.

SEQ ID NO: 125

This shows the amino acid sequence of the fusion protein of orphan receptor GPR21 and GFP-1.

SEQ ID NO: 126

This shows the amino acid sequence of the fusion protein of orphan receptor GPR48 and GFP-1.

SEQ ID NO: 127

This shows the amino acid sequence of the fusion protein of orphan receptor SNORF1 and GFP-1.

SEQ ID NO: 128

This shows the amino acid sequence of the fusion protein of orphan receptor BA12 and GFP-1.

SEQ ID NO: 129

This shows the amino acid sequence of the fusion protein of orphan receptor MAS and GFP-1.

SEQ ID NO: 130

This shows the amino acid sequence of the fusion protein of orphan receptor OT7T009 and GFP-1.

SEQ ID NO: 131

This shows the amino acid sequence of the fusion protein of orphan receptor TGR34 and GFP-1.

SEQ ID NO: 132

This shows the base sequence of DNA encoding the fusion protein of orphan receptor hBL5 and GFP-1.

SEQ ID NO: 133

This shows the base sequence of DNA encoding the fusion protein of orphan receptor h7TBA62 and GFP-1.

SEQ ID NO: 134

This shows the base sequence of DNA encoding the fusion protein of orphan receptor 14273 and GFP-1.

SEQ ID NO: 135

This shows the base sequence of DNA encoding the fusion protein of orphan receptor EMR3 and GFP-1.

SEQ ID NO: 136

This shows the base sequence of DNA encoding the fusion protein of orphan receptor GPR15 and GFP-1.

SEQ ID NO: 137

This shows the base sequence of DNA encoding the fusion protein of orphan receptor GPR31 and GFP-1.

SEQ ID NO: 138

This shows the base sequence of DNA encoding the fusion protein of orphan receptor GPRC5B and GFP-1.

SEQ ID NO: 139

This shows the base sequence of DNA encoding the fusion protein of orphan receptor PSEC0142 and GFP-1.

SEQ ID NO: 140

This shows the base sequence of DNA encoding the fusion protein of orphan receptor HE6 and GFP-1.

SEQ ID NO: 141

This shows the base sequence of DNA encoding the fusion protein of orphan receptor GPR61 and GFP-1.

SEQ ID NO: 142

This shows the base sequence of DNA encoding the fusion protein of orphan receptor TGR9 and GFP-1.

SEQ ID NO: 143

This shows the base sequence of DNA encoding the fusion protein of orphan receptor TGR24 and GFP-1.

SEQ ID NO: 144

This shows the base sequence of DNA encoding the fusion protein of orphan receptor ZGPR1 and GFP-1.

SEQ ID NO: 145

This shows the base sequence of DNA encoding the fusion protein of orphan receptor EMR1 and GFP-1.

SEQ ID NO: 146

This shows the base sequence of DNA encoding the fusion protein of orphan receptor GPR25 and GFP-1.

SEQ ID NO: 147

This shows the base sequence of DNA encoding the fusion protein of orphan receptor GPR55 and GFP-1.

SEQ ID NO: 148

This shows the base sequence of DNA encoding the fusion protein of orphan receptor AXOR14 and GFP-1.

SEQ ID NO: 149

This shows the base sequence of DNA encoding the fusion protein of orphan receptor TM7SF1 and GFP-1.

SEQ ID NO: 150

This shows the base sequence of DNA encoding the fusion protein of orphan receptor PSP24B and GFP-1.

SEQ ID NO: 151

This shows the base sequence of DNA encoding the fusion protein of orphan receptor SREB3 and GFP-1.

SEQ ID NO: 152

This shows the base sequence of DNA encoding the fusion protein of orphan receptor TGR37 and GFP-1.

SEQ ID NO: 153

This shows the base sequence of DNA encoding the fusion protein of orphan receptor H963 and GFP-1.

SEQ ID NO: 154

This shows the base sequence of DNA encoding the fusion protein of orphan receptor GPR87 and GFP-1.

SEQ ID NO: 155

This shows the base sequence of DNA encoding the fusion protein of orphan receptor GPR91 and GFP-1.

SEQ ID NO: 156

This shows the base sequence of DNA encoding the fusion protein of orphan receptor PNR and GFP-1.

SEQ ID NO: 157

This shows the base sequence of DNA encoding the fusion protein of orphan receptor TGR29 and GFP-1.

SEQ ID NO: 158

This shows the base sequence of DNA encoding the fusion protein of orphan receptor TGR3 6 and GFP-1.

SEQ ID NO: 159

This shows the base sequence of DNA encoding the fusion protein of orphan receptor H9 and GFP-1.

SEQ ID NO: 160

This shows the base sequence of DNA encoding the fusion protein of orphan receptor TGR18 and GFP-1.

SEQ ID NO: 161

This shows the base sequence of DNA encoding the fusion protein of orphan receptor TGR19 and GFP-1.

SEQ ID NO: 162

This shows the base sequence of DNA encoding the fusion protein of orphan receptor AM-R and GFP-1.

SEQ ID NO: 163

This shows the base sequence of DNA encoding the fusion protein of orphan receptor GPR19 and GFP-1.

SEQ ID NO: 164

This shows the base sequence of DNA encoding the fusion protein of orphan receptor GPR45 and GFP-1.

SEQ ID NO: 165

This shows the base sequence of DNA encoding the fusion protein of orphan receptor GPRC5D and GFP-1.

SEQ ID NO: 166

This shows the base sequence of DNA encoding the fusion protein of orphan receptor LGR6 and GFP-1.

SEQ ID NO: 167

This shows the base sequence of DNA encoding the fusion protein of orphan receptor RUP3 and GFP-1.

SEQ ID NO: 168

This shows the base sequence of DNA encoding the fusion protein of orphan receptor TGR14 and GFP-1.

SEQ ID NO: 169

This shows the base sequence of DNA encoding the fusion protein of orphan receptor TPRA40 and GFP-1.

SEQ ID NO: 170

This shows the base sequence of DNA encoding the fusion protein of orphan receptor GPR22 and GFP-1.

SEQ ID NO: 171

This shows the base sequence of DNA encoding the fusion protein of orphan receptor GPR52 and GFP-1.

SEQ ID NO: 172

This shows the base sequence of DNA encoding the fusion protein of orphan receptor FLH2882 and GFP-1.

SEQ ID NO: 173

This shows the base sequence of DNA encoding the fusion protein of orphan receptor SNORF36 and GFP-1.

SEQ ID NO: 174

This shows the base sequence of DNA encoding the fusion protein of orphan receptor MRG and GFP-1.

SEQ ID NO: 175

This shows the base sequence of DNA encoding the fusion protein of orphan receptor SREB2 and GFP-1.

SEQ ID NO: 176

This shows the base sequence of DNA encoding the fusion protein of orphan receptor GPR12 and GFP-1.

SEQ ID NO: 177

This shows the base sequence of DNA encoding the fusion protein of orphan receptor GPR30 and GFP-1.

SEQ ID NO: 178

This shows the base sequence of DNA encoding the fusion protein of orphan receptor GPR82 and GFP-1.

SEQ ID NO: 179

This shows the base sequence of DNA encoding the fusion protein of orphan receptor RECAP and GFP-1.

SEQ ID NO: 180

This shows the base sequence of DNA encoding the fusion protein of orphan receptor HB954 and GFP-1.

SEQ ID NO: 181

This shows the base sequence of DNA encoding the fusion protein of orphan receptor RDC1 and GFP-1.

SEQ ID NO: 182

This shows the base sequence of DNA encoding the fusion protein of orphan receptor TGR6 and GFP-1.

SEQ ID NO: 183

This shows the base sequence of DNA encoding the fusion protein of orphan receptor A-2 and GFP-1.

SEQ ID NO: 184

This shows the base sequence of DNA encoding the fusion protein of orphan receptor JEG18 and GFP-1.

SEQ ID NO: 185

This shows the base sequence of DNA encoding the fusion protein of orphan receptor GPR17 and GFP-1.

SEQ ID NO: 186

This shows the base sequence of DNA encoding the fusion protein of orphan receptor GPR35 and GFP-1.

SEQ ID NO: 187

This shows the base sequence of DNA encoding the fusion protein of orphan receptor GPRC5C and GFP-1.

SEQ ID NO: 188

This shows the base sequence of DNA encoding the fusion protein of orphan receptor HM74 and GFP-1.

SEQ ID NO: 189

This shows the base sequence of DNA encoding the fusion protein of orphan receptor RPE and GFP-1.

SEQ ID NO: 190

This shows the base sequence of DNA encoding the fusion protein of orphan receptor TGR 13 and GFP-1.

SEQ ID NO: 191

This shows the base sequence of DNA encoding the fusion protein of orphan receptor TGR27 and GFP-1.

SEQ ID NO: 192

This shows the base sequence of DNA encoding the fusion protein of orphan receptor DEZ and GFP-1.

SEQ ID NO: 193

This shows the base sequence of DNA encoding the fusion protein of orphan receptor ratGPR1 and GFP-1.

SEQ ID NO: 194

This shows the base sequence of DNA encoding the fusion protein of orphan receptor GPR3 and GFP-1.

SEQ ID NO: 195

This shows the base sequence of DNA encoding the fusion protein of orphan receptor GPR6 and GFP-1.

SEQ ID NO: 196

This shows the base sequence of DNA encoding the fusion protein of orphan receptor RAIG1 and GFP-1.

SEQ ID NO: 197

This shows the base sequence of DNA encoding the fusion protein of orphan receptor TGR2-1 and GFP-1.

SEQ ID NO: 198

This shows the base sequence of DNA encoding the fusion protein of orphan receptor TGR2-2 and GFP-1.

SEQ ID NO: 199

This shows the base sequence of DNA encoding the fusion protein of orphan receptor TGR21 and GFP-1.

SEQ ID NO: 200

This shows the base sequence of DNA encoding the fusion protein of orphan receptor GPR56 and GFP-1.

SEQ ID NO: 201

This shows the base sequence of DNA encoding the fusion protein of orphan receptor KIAA0758 and GFP-1.

SEQ ID NO: 202

This shows the base sequence of DNA encoding the fusion protein of orphan receptor RE2 and GFP-1.

SEQ ID NO: 203

This shows the base sequence of DNA encoding the fusion protein of orphan receptor P40 and GFP-1.

SEQ ID NO: 204

This shows the base sequence of DNA encoding the fusion protein of orphan receptor GPR27 and GFP-1.

SEQ ID NO: 205

This shows the base sequence of DNA encoding the fusion protein of orphan receptor HG38 and GFP-1.

SEQ ID NO: 206

This shows the base sequence of DNA encoding the fusion protein of orphan receptor DRR1 and GFP-1.

SEQ ID NO: 207

This shows the base sequence of DNA encoding the fusion protein of orphan receptor TGR12 and GFP-1.

SEQ ID NO: 208

This shows the base sequence of DNA encoding the fusion protein of orphan receptor TGR11 and GFP-1.

SEQ ID NO: 209

This shows the base sequence of DNA encoding the fusion protein of orphan receptor TGR15 and GFP-1.

SEQ ID NO: 210

This shows the base sequence of DNA encoding the fusion protein of orphan receptor TGR8 and GFP-1.

SEQ ID NO: 211

This shows the base sequence of DNA encoding the fusion protein of orphan receptor GPR20 and GFP-1.

SEQ ID NO: 212

This shows the base sequence of DNA encoding the fusion protein of orphan receptor TGR10 and GFP-1.

SEQ ID NO: 213

This shows the base sequence of DNA encoding the fusion protein of orphan receptor TGR30 and GFP-1.

SEQ ID NO: 214

This shows the base sequence of DNA encoding the fusion protein of orphan receptor GPR1 8 and GFP-1.

SEQ ID NO: 215

This shows the base sequence of DNA encoding the fusion protein of orphan receptor TGR25 and GFP-1.

SEQ ID NO: 216

This shows the base sequence of DNA encoding the fusion protein of orphan receptor GPR23 and GFP-1.

SEQ ID NO: 217

This shows the base sequence of DNA encoding the fusion protein of orphan receptor P2Y10 and GFP-1.

SEQ ID NO: 218

This shows the base sequence of DNA encoding the fusion protein of orphan receptor GPR37 and GFP-1.

SEQ ID NO: 219

This shows the base sequence of DNA encoding the fusion protein of orphan receptor ET(B)R-LP-2 and GFP-1.

SEQ ID NO: 220

This shows the base sequence of DNA encoding the fusion protein of orphan receptor FPRL2 and GFP-1.

SEQ ID NO: 221

This shows the base sequence of DNA encoding the fusion protein of orphan receptor GPR32 and GFP-1.

SEQ ID NO: 222

This shows the base sequence of DNA encoding the fusion protein of orphan receptor dj287G14.2 and GFP-1.

SEQ ID NO: 223

This shows the base sequence of DNA encoding the fusion protein of orphan receptor BRS-3 and GFP-1.

SEQ ID NO: 224

This shows the base sequence of DNA encoding the fusion protein of orphan receptor GPR39 and GFP-1.

SEQ ID NO: 225

This shows the base sequence of DNA encoding the fusion protein of orphan receptor 63A2 and GFP-1.

SEQ ID NO: 226

This shows the base sequence of DNA encoding the fusion protein of orphan receptor GPR84 and GFP-1.

SEQ ID NO: 227

This shows the base sequence of DNA encoding the fusion protein of orphan receptor GPR21 and GFP-1.

SEQ ID NO: 228

This shows the base sequence of DNA encoding the fusion protein of orphan receptor GPR48 and GFP-1.

SEQ ID NO: 229

This shows the base sequence of DNA encoding the fusion protein of orphan receptor SNORF1 and GFP-1.

SEQ ID NO: 230

This shows the base sequence of DNA encoding the fusion protein of orphan receptor BA12 and GFP-1.

SEQ ID NO: 231

This shows the base sequence of DNA encoding the fusion protein of orphan receptor MAS and GFP-1.

SEQ ID NO: 232

This shows the base sequence of DNA encoding the fusion protein of orphan receptor OT7T009 and GFP-1.

SEQ ID NO: 233

This shows the base sequence of DNA encoding the fusion protein of orphan receptor TGR34 and GFP-1.

The transformant Escherichia coli JM109/pCR4-hTGR5 obtained in REFERENCE EXAMPLE 1 has been on deposit with International Patent Organisms Depository, National Institute of Advanced Industrial Science and Technology (formerly, National Institute of Bioscience and Human-Technology (NIBH), Ministry of Economics, Trade and Industry) located at Central 6, 1-1-1 Higashi, Tsukuba, Ibaraki (post code: 305-8566), as the Accession Number FERM BP-7114 since Apr. 3, 2000, and with Institute for Fermentation (IFO), located at 2-17-85 Juso-honmachi, Yodogawa-ku, Osaka-shi, Osaka (post code: 532-8686), as the Accession Number IFO 16410 since March 23, 2000.

### EXAMPLES

The present invention is described in detail below with reference to EXAMPLES and REFERENCE EXAMPLES, but is not deemed to limit the scope of the present invention thereto. The gene manipulation procedures using Escherichia coli were performed according to the methods described in the Molecular Cloning.

### Reference Example 1

Cloning of cDNA Encoding G Protein-Coupled Receptor Protein of Human Spleen and Determination of the Base Sequence

Using human spleen-derived cDNA (Clontech) as a template and two primers, namely, primer 1 (SEQ ID NO: 11) and primer 2 (SEQ ID NO: 12), PCR was carried out. The reaction solution in the above reaction comprised of 1/10 volume of the cDNA described above as a template, 1/50 volume of Advantage-GC2 Polymerase Mix (Clontech), 0.5 μM each of primer 1 (SEQ ID NO: 11) and primer 2 (SEQ ID NO: 12), 200 μM of dNTPs, 1/5 volume of a buffer attached to the enzyme product and 1/5 volume of GC Melt to make the total volume 20 11. The PCR reaction was carried out by reacting at 94° C. for 5 minutes, then repeating 30 times a cycle set to include 94° C. for 30 seconds, 60° C. for 30 seconds and 68° C. for 2 minutes, and finally conducting extension at 68° C. for 5 minutes. The PCR product was subcloned to plasmid vector pCR4 (Invitrogen) following the instructions attached to the TA Cloning Kit (Invitrogen). The plasmid was then transfected to Escherichia coli JM109, and the clones containing the cDNA of the PCR product were selected on LB agar plates containing ampicillin. As a result of analysis for the sequence of each clone, cDNA sequence encoding a novel G protein-coupled receptor protein was obtained (SEQ ID NO: 10). The novel G protein-coupled receptor protein containing the amino acid sequence (SEQ ID NO: 9) deduced from this cDNA sequence was named TGR5. Also, the transformant containing DNA represented by SEQ ID NO: 10 was named Escherichia coli JM109/pCR4-hTGR5.

### Reference Example 2

Detection of Reporter Activation with Cholesterol Metabolism-Related Substance in Human HEK293 Cells wherein TGR5 was Expressed Transiently

The TGR5-specific stimulation activity by cholesterol metabolism-related substance was detected using as an indicator the expression level of a reporter gene product (luciferase) produced by expression induction of CRE promoter.

Human-derived HEK293 cells were suspended in growth redium (DMEM (Dulbecco's Modified Eagle Medium) (Gibco BRL) supplemented with 10% fetal cow serum (Gibco BRL)). The suspension adjusted to a concentration of 1×10⁵ cells/well was plated on a collagen-coated black well 96-well plate (Becton Dickinson, Inc.).

After culturing overnight at 37° C. under the 5% CO₂ condition, the TGR5 gene was inserted into an expression vector for animal cell pAKKO-111H (the same plasmid vector as pAKKO-1.111H described in Biochem. Biophys. Acta, Hinuma, S. et al., 1219, 251-259, 1994) by publicly known methods, together with a reporter gene-containing plasmid pCRE-Luc (Clontech). The thus prepared TGR5 expression vector plasmid or intact pAKKO-111H containing no TGR5 gene was transfected to the cells as described below.

By mixing OPTI-MEM-I (Gibco BRL, Inc.) and Lipofectamine™ 2000 reagent (Gibco BRL, Inc.) in 24:1, a Lipofectamine dilution was prepared. Also, OPTI-MEM-I, TGR5 expression vector plasmid or the intact vector plasmid (240 ng/μl) and pCRE-Luc (240 ng/μl) were mixed in 24:0.9:0.1 to prepare a DNA dilution. The Lipofectamine dilution and the DNA dilution were mixed in equal volumes and the mixture was allowed to stand for 20 minutes at room temperature to produce the DNA-Lipofectamine complex. Then, 25 μl of the solution was added to the HEK293 cell-incubated plate described above, followed by further culturing overnight at 37° C. under the 5% CO₂ condition.

The transfected HEK293 cells were washed with an assay medium (DMEM supplemented with 0.1% bovine serum albumin) and lithocholic acid (Wako Pure Chemical Industries, Inc.) and progesterone (Wako Pure Chemical Industries, Inc.) diluted with the assay medium to 2×10⁻⁵ M were added to the plate, which was then incubated for 4 hours at 37° C. under the 5% CO₂ condition. The culture supernatant was discarded and 50 μl of a substrate for assaying the luciferase activity or PicaGene LT 2.0 (Toyo Ink Mfg. Co., Ltd.) was added. The fluorescence level of luciferase was measured by using a plate reader (ARVO sx multi-label counter, Wallac, Inc.).

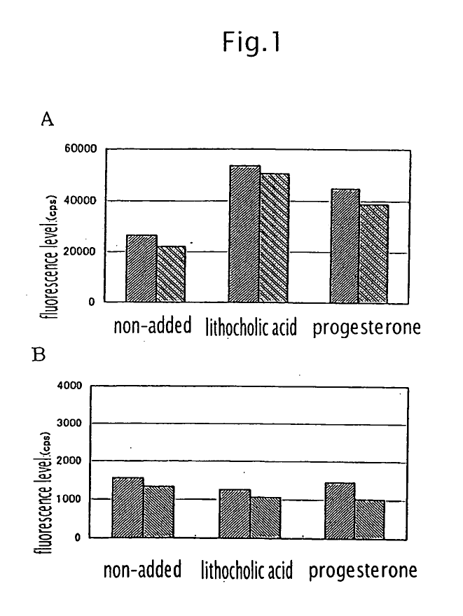

As a result, an increased luciferase activity by lithocholic acid and progesterone was noted specifically to the HEK293 cells transfected with TGR5 gene having the base sequence represented by SEQ ID NO: 10 (FIG. 1).

### Reference Example 3

Transfection of G Protein-Coupled Receptor Protein-Expressed Plasmid and Reporter Plasmid to Host Cells

Using expression plasmids for animal cells inserted with various G protein-coupled receptor protein cDNAs prepared by publicly known methods, i.e., thyrotropin releasing hormnone receptor (TRHR), neuromedin U receptor (FM-3 and TGR-1), prolactin releasing factor receptor (hGR3), apelin receptor (APJ), etc., Escherichia coli JM109 was transfected and the colonies obtained were isolated and cultured. Thereafter, plasmid was prepared using QIAGEN Plasmid Maxi Kit (Qiagen). Furthermore, the reporter plasmid of pCRE-Luc (Clontech) ligated with a luciferase gene as a reporter at the downstream of cAMP response element (CRE) was prepared in a similar manner.

Human HEK293 cells as host cells for transfecting the G protein-coupled receptor protein and the reporter plasmid thereto were plated on a type I collagen-coated 96-well black plate (Becton Dickinson, Inc.) in 100,000 cells/well in a culture solution volume of 100 μl, followed by culturing overnight. In the same way, CHO (dhfr-) cells were transformed by pAKKO-111H and the resulting CHO-mock cells were plated on a 96-well black plate of Costar, Inc. in 40,000 cells/well in a culture solution volume of 100 μl, followed by culturing overnight. In the both cells, DMEM (Gibco BRL, Inc.) supplemented with 10% fetal calf serum alone was used as a medium for plate culture.

Each of the plasmids above was diluted to a concentration of 240 ng/μl, and the dilution was added to 240 μl of Opti-MEM-I (Gibco BRL, Inc.) in a proportion of 9 μl of the G protein-coupled receptor protein expression plasmid and 1 μl of the reporter plasmid. The mixture was mixed with 240 μl of Opti-MEM-I added with 10 μl of Lipofectamine 2000 (Gibco BRL, Inc.) in equal volumes to form the liposome and plasmid complex, in accordance with the procedures in the instruction manuals attached to Lipofectamine 2000. Also, for carrying out efficient screening, 5 μl each of the 3 receptor expression plasmids was added in a concentration of 240 ng/μl and the mixtures having the same proportion of the other reagents as described above were prepared. The mixtures were added to the culture solution of HEK293 or CHO-mock cells in 25 μl each/well and incubated at 37° C. overnight to introduce the plasmids. In the CHO-mock cells, the culture solution was replaced by an assay buffer (DMEM supplemented with 0.1% bovine serum albumin) 4 hours after the plasmid addition to make serum-free.

### Reference Example 4

Detection of Ligand Activity by Reporter Assay

In the HEK293 cells, the culture solution was replaced by the assay buffer of REFERENCE EXAMPLE 3 an hour before the assaying and pre-incubated. A solution of a ligand or a ligand candidate compound in the assay buffer was prepared and added to the HEK293 cells or CHO-mock cells prepared in REFERENCE EXAMPLE 3. Also, an assay was conducted in a similar manner under the conditions where forskolin was added to the assay buffer in the final concentration of 2 μM. After the ligand or the test compound was added, incubation was carried out for 4 hours to induce promotion or suppression of the transcription/translation of the reporter gene derived from intracellular signal transduction caused by the agonist activity of ligand mediated by the receptor. After the incubation was completed, the assay buffer in each well was removed and 50 μl each of luminescent substrate PicaGene LT 2.0 (Toyo Ink Mfg. Co., Ltd.) was added to the well. After the cells were lysed and thoroughly mixed with the substrate, the luminescent level corresponding to the expression induction level of the reporter gene in each well was measured on the plate reader described in REFERENCE EXAMPLE 2.

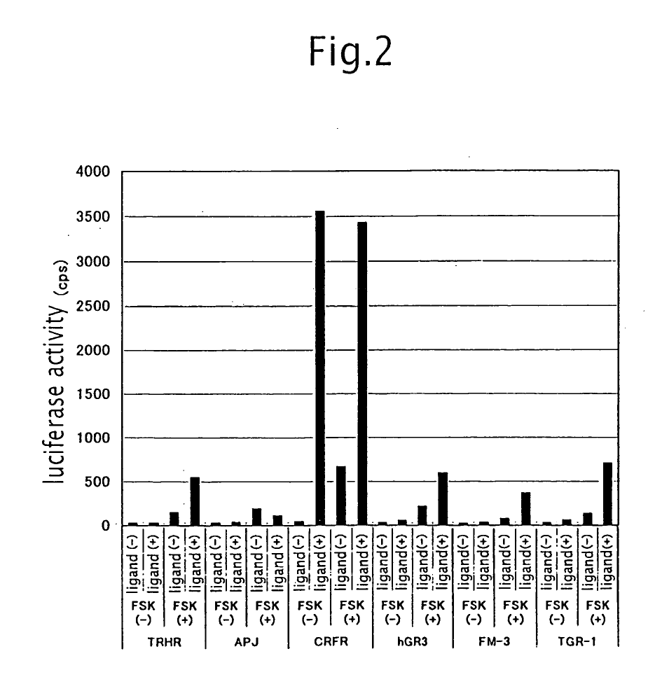

Using expression plasmids in which various G protein-coupled receptor protein cDNAs were inserted in accordance with the procedures described in REFERENCE EXAMPLES 3 and 4, the expression induction of the reporter gene by ligand stimulation was assayed in HEK293 cells. In CRFR coupled to GS as a subunit of G protein for transduction of signals into cells, activation of the reporter gene due to the ligand addition was detected under both conditions in the absence of and in the presence of forskolin. In APJ coupled to inhibitory Gi, suppressed expression of the reporter gene due to the ligand addition was detected under the condition that forskolin was added. Further in the receptors TRHR, FM-3 and TGR-1 coupled to Gq, promoted expression of the reporter gene was detected under the condition that forskolin was added. Also in the receptor hGR3 coupled to both Gq and Gi, promoted expression of the reporter gene was detected as well, under the condition that forskolin was added (FIG. 2).

### Reference Example 5

Reporter Assay using Inhibitory G Protein a Subunit Gi-Expressed Plasmid

Inhibitory G protein α subunit (Gi)-expressed plasmid was prepared in a similar manner to the G protein receptor-expressed plasmid shown in REFERENCE EXAMPLE 3 (herein Gi may be any kind irrespective of any animal species). This plasmid, the receptor-expressed plasmid and the reporter plasmid were added to 240 μl of Opti-MEM-I in the proportion of 3 μl, 7 μl and 1 μl and DNA was introduced into HEK293 or CHO-mock cells otherwise under the same conditions as in EXAMPLE 2. The mixing ratio of these 3 plasmids is from 1 to 6 μl of Gi, preferably from 1 to 3 μl, when the total amount is made 11 μl. These plasmids were assayed in accordance with the procedures of EXAMPLE 3 and the ligand activities were detected.

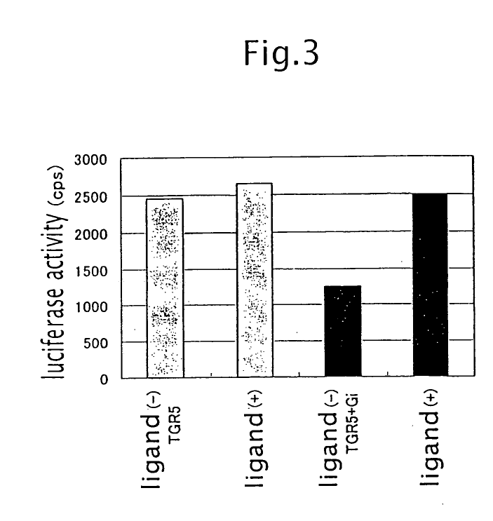

That is, the response of TGR5 to lithocholic acid in the co-presence of Gi was detected. As a result, Gi was co-expressed together with TGR5 in the assay for G protein receptor TGR5 using the CHO-mock cells, whereby the luciferase activity added with no ligand (ligand (−)) could be markedly reduced, which enabled to detect the increase in activity by the ligand (lithocholic acid, 2×10⁻⁵ M, ligand (+)) (FIG. 3).

### Example 1

Internalization of the TGR5-GFP Fusion Protein Expressed on CHO Cells by the Addition of Taurolithocholic Acid

An expression plasmid was constructed to express the fusion protein in which TGR5 was fused at the C terminus with Green Fluorescent Protein (GFP) isolated from jelly fish Aequorea victoria. In this case, a fragment excised from expression vector pQBI25 (TaKaRa Shuzo) for GFP was used as cDNA (SEQ ID NO: 2) of GFP. In the cDNA of TGR5, its termination codon was corrected by PCR to the recognition sequence of restriction enzyme NheI, and the cDNA fragment of GFP was ligated thereto, which was inserted into expression vector pAKKO-111H described in EXAMPLE 1. The expression vector plasmid for the thus obtained fusion protein of TGR5 and GFP (hereinafter TGR5-GFP) was transfected to CHO-mock cells by the following procedures. The CHO-mock cells were suspended in growth medium [DMEM (Dulbecco's Modified Eagle Medium) (GIBCO BRL, Inc.) supplemented with 10% fetal calf serum (GIBCO BRL, Inc.)] and plated on a Lab-TekII coverglass chamber (Nalgen Nunc, Inc.) with 4 chamber compartments in a concentration of 0.6×10⁵ cells/chamber. After culturing overnight at 37° C. under the 5% CO₂ condition, transfection was effected. For the transfection, Lipofectamine™ 2000 reagent (GIBCO BRL, Inc.) was used. First, 2 μl of Lipofectamine™ 2000 reagent was mixed with 50 μl of OPTI-MEM-I (GIBCO BRL, Inc.). After allowing to stand for 5 minutes, the mixture wasgmixed with a solution mixture of 0.48 μg of DNA and 50 μl of OPTI-MEM-1. The mixture was left to stand at room temperature for 20 minutes, thereby to form the DNA-lipofectamine complex. After 100 μl of the mixture was added to the chamber above where the CHO cells were incubated, incubation was conducted overnight at 37° C. under the 5% CO₂ condition. The medium was replaced by medium for confocal laser scanning microscopic observation [suspension of 0.1% bovine albumin (Essentially Fatty Acid Free (GIBCO BRL, Inc.) in Hanks' Balanced Salt Solution (GIBCO BRL, Inc.))] and the fluorescent image of GFP was observed on a confocal laser scanning microscope (Leica). In this case, GFP was excited at 488 nm.

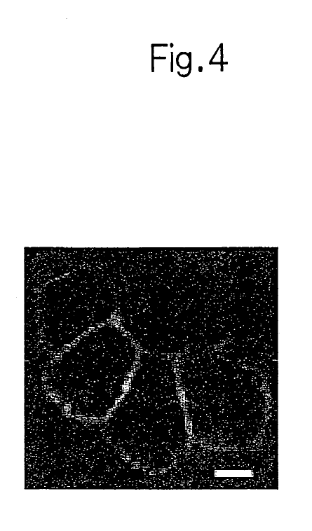

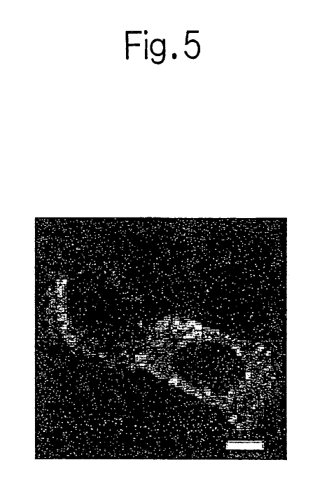

As a result, the TGR5-GFP fluorescent protein was observed in the cell membrane (FIG. 4). Taurolithocholic acid was added to the cells to have 10⁻⁵ M in the medium. It was found that 30 minutes after, the fluorescence of GFP was moved to the cytoplasm, not in the cell membrane (FIG. 5). This indicates that TGR5 is a G protein-coupled receptor and at the same time, TGR5 moves to the cytoplasm, namely, internalized, in response to taurolithocholic acid.

### Example 2

Expression of the Fusion Protein of Parathyroid Hormone Receptor (PTH-R) and GFP in Pancreas β Cell Line RINm5F by Transient Expression

Expression vector was prepared in a manner similar to EXAMPLE 1 by inserting an expression plasmid for expressing the fusion protein of human PTH-R (SEQ ID NO: 13) and GFP at the C terminus into expression vector pAKKO-111H described in REFERENCE EXAMPLE 2. The expression vector plasmid for the thus obtained fusion protein of PTH-R and GFP (hereinafter PTH-GFP) was transfected to RINm5F cells by the following procedures. The RINm5F cells were suspended in growth medium [RPMI1640 (GIBCO BRL, Inc.) supplemented with 10% fetal calf serum (GIBCO BRL, Inc.) treated by Charcoal/Dextran] and plated on a Lab-TekII coverglass chamber (Nalgen Nunc, Inc.) with 8 chamber compartments in a concentration of 0.3×10⁵ cells/chamber. After culturing overnight at 37° C. under the 5% CO₂ condition, transfection was effected. For the transfection, Lipofectamine™ 2000 reagent (GIBCO BRL, Inc.) was used. First, 3.3 μl of Lipofectamine™ 2000 reagent was mixed with 80 μl of OPTI-MEM-I (GIBCO BRL, Inc.). After allowing to stand for 5 minutes, the mixture was mixed with a solution mixture of 0.72 Hg of DNA and 80 μl of OPTI-MEM-I. The mixture was left to stand at room temperature for 20 minutes, thereby to form the DNA-lipofectamine complex. After 160 μl of the solution mixture was added to the chamber above where the RINm5F cells were incubated, the cells were incubated overnight at 37° C. under the 5% CO₂ condition. Confocal laser scanning microscopic observation was performed in a manner similar to EXAMPLE 1.

As a result, it was observed that the PTH-GFP fusion protein was expressed in the cell membrane.

### Example 3

Expression of the GPCR and GFP Fusion Protein using Insulin II Promoter

In a manner similar to EXAMPLE 1, a fragment of human GPR40 (SEQ ID NO: 15) ligated with GFP at the C terminus was inserted at the downstream of mouse insulin II promoter cloned from mouse genome to prepare an expression vector for expressing the fusion protein of GPR40 and GFP (hereinafter GPR40-GFP). The expression vector plasmid for the thus obtained GPR40-GFP was transfected to MIN6 cells by the following procedures. The MIN6 cells were suspended in growth medium [DMEM (containing 4.5 g/l Glucose) (Invitrogen, Inc.) supplemented with 15% fetal calf serum (Trace, Inc.) in the final concentration, 55 μM of 2-mercaptoethanol (Invitrogen, Inc.) and 20 mM HEPES (Dainippon Pharmaceutical Co., Ltd.)] and plated on a Lab-TekII coverglass chamber (Nalgen Nunc, Inc.) with 4 chamber compartments in a concentration of 1.2×10⁵ cells/chamber. After culturing for 2 nights at 37° C. under the 5% CO₂ condition, transfection was effected. For the transfection, Lipofectamine™ 2000 reagent (GIBCO BRL, Inc.) was used. First, 4 μl of Lipofectamine™ 2000 reagent was mixed with 100 μl of Opti-MEM (Invitrogen, Inc.). After allowing to stand for 5 minutes, the mixture was mixed with a solution mixture of 2 μg of DNA and 100 μl of Opti-MEM medium. The mixture was left to stand at room temperature for 20 minutes, thereby to form the DNA-lipofectamine complex. After 100 μl of the solution mixture was added to the chamber above where the MIN6 cells were incubated, the cells were incubated for 4 hours at 37° C. under the 5% CO₂ condition. Then, the medium was replaced by 400 μl of a fresh growth medium followed by further incubation overnight. Confocal laser scanning microscopic observation was made as in EXAMPLE 1.

As a result, the GPR40-GFP fluorescent protein was observed in the cell membrane of MIN6 cells.

### Example 4

Preparation of Transformants Capable of Individually Expressing 102 Kinds of Fusion Proteins

Expression vector plasmids of 102 kinds containing the respective DNAs (SEQ ID NO: 132 through SEQ ID NO: 233) encoding the fusion proteins of 102 kinds of the respective receptor proteins for which ligands have not been identified and GFP consisting of the amino acid sequence represented by SEQ ID NO: 1 or its modified amino acid sequences were prepared, and transfected into CHO-mock cells. These CHO cells were incubated as in EXAMPLE 1. It was observed that the fusion proteins were expressed in the CHO-mock cells.

Industrial Applicability

Since various cell lines can be used, the methods of the present invention for determining ligands to receptor proteins for which ligands have not been identified are simple and can be performed in a short period of time.

1. A method of determining a ligand to a receptor protein for which a ligand has not been identified, which comprises using a fusion protein of the receptor protein and a fluorescent protein.

2. The ligand determination method according to claim 1, wherein the fusion protein of a receptor protein for which a ligand has not been identifyd and GFP is used.

3. The ligand determination method according to claim 1, wherein a cell expressed with the fusion protein of a receptor protein for which a ligand has not been identified and GFP or a membrane fraction of the cell is brought in contact with a test compound.

4. The ligand determination method according to claim 1, which comprises assaying (1) an activity that accelerates or suppresses arachidonic acid release, acetylcholine release, intracellular Ca²⁺ release, intracellular cAMP production, intracellular cGMP production, inositol phosphate production, changes in cell membrane potential, phosphorylation of intracellular proteins, activation of c-fos or pH reduction, (2) MAP kinase activation, (3) transcription factor activation, (4) diacylglycerol production, (5) opening or closing of ion channels on a cell membrane, (6) apoptosis induction, (7) morphological changes in cells, (8) transport of the fusion protein from cell membrane to cytoplasm, (9) low molecular weight G protein activation, (10) cell division-promoting activity or (11) DNA synthesis-promoting activity.

5. The ligand determination method according to claim 1, wherein transport of the fusion protein from cell membrane to cytoplasm is assayed.

6. The ligand determination method according to claim 5, wherein transport of the fusion protein from cell membrane to cytoplasm is assayed by observing the fluorescence of GFP.

7. The ligand determination method according to claim 1, which comprises bringing a cell capable of expressing the fusion protein of a receptor protein for which a ligand has not been identified the receptor protein and a fluorescent protein and containing a plasmid ligated with a DNA encoding a reporter protein at the downstream of cAMP response element/promoter in contact with a test compound and assaying the activity of the reporter protein.

8. The method according to claim 7, which comprises culturing a cell containing (1) a plasmid containing a DNA encoding the fusion protein of a receptor protein for which a ligand has not been identified and a fluorescent protein and (2) a plasmid ligated with a DNA encoding a reporter protein at the downstream of cAMP response element/promoter, and bringing the cell in contact with a test compound and assaying the activity of the reporter protein.

9. The ligand determination method according to claim 2, wherein a cell capable of expressing the fusion protein of a receptor protein for which a ligand has not been identified and GFP and containing a plasmid ligated with a DNA encoding a reporter protein at the downstream of cAMP response element/promoter is brought in contact with a test compound and assaying the activity of the reporter protein.

10. The ligand determination method according to claim 9, which comprises culturing a cell containing (1) a plasmid containing a DNA encoding the fusion protein of a receptor protein for which a ligand has not been identified and GFP and (2) a plasmid ligated with a DNA encoding a reporter protein at the downstream of cAMP response element/promoter, bringing the cell in contact with a test compound and assaying the activity of the reporter protein.

11. The method according to claim 1, wherein the receptor protein is a G protein-coupled receptor protein.

12. The method according to claim 1, wherein GFP is a protein containing the same or substantially the same amino acid sequence as the amino acid sequence represented by SEQ ID NO: 1, SEQ ID NO: 3, SEQ ID NO: 5 or SEQ ID NO: 7.

13. The method according to claim 7, wherein the promoter is a TATA-like sequence.

14. The method according to claim 7, wherein the reporter protein is luciferase.

15. The method according to claim 7, wherein the plasmid is a plasmid ligated with a TATA-like promoter and a gene encoding the reporter protein at the downstream of cAMP response element.

16. The method according to claim 7, wherein the cell has expressed at least two receptor proteins for which ligands have not been identified.

17. The method according to claim 7, wherein the cell further contains a plasmid containing a gene encoding an inhibitory G protein a subunit Gi.

18. The method according to claim 7, wherein forskolin is further added.

19. The method according to claim 16, wherein the at least two receptor proteins have similar characteristics.

20. The method according to claim 19, wherein the similar characteristics are the basic expression level of the reporter protein and/or the expression level of the reporter protein when fdrskolin is added.

21. The method according to claim 16, which comprises measuring the basic expression level of the reporter protein when the at least two receptor proteins are expressed individually and/or the expression level of the reporter protein by the addition of forskolin, and being expressed in combination the at leas two receptor proteins having an equivalent expression level of the reporter protein.

22. A fusion protein of a receptor protein for which a ligand has not been identified and a fluorescent protein, or a salt thereof.

23. The fluorescent protein or a salt thereof according to claim 22, wherein the fluorescent protein is GFP.

24. A DNA containing a DNA encoding the fusion protein according to claim 22.

25. A recombinant vector containing the DNA according to claim 22.

26. A transfornant transformed with the recombinant vector according to claim 25.

27. Use of a fluorescent protein to determine a ligand to a receptor protein for which a ligand has not been identified.

28. Use of GFP to determine a ligand to a receptor protein for which a ligand has not been identified.
